(19) United States
(12) Patent Application Publication
(10) Pub. No.: US 2017/0218389 A1
(43) Pub. Date: Aug. 3, 2017

(54) METHODS AND COMPOSITIONS FOR STOMATA REGULATION, PLANT IMMUNITY, AND DROUGHT TOLERANCE

(71) Applicant: The Samuel Roberts Noble Foundation, Inc., Ardmore, OK (US)

(72) Inventors: Kirankumar Mysore, Ardmore, OK (US); Amita Kaundal, Ardmore, OK (US); Seonghee Lee, Ardmore, OK (US)

(21) Appl. No.: 15/404,071

(22) Filed: Jan. 11, 2017

Related U.S. Application Data
(60) Provisional application No. 62/278,881, filed on Jan. 14, 2016.

(51) Int. Cl. C12N 15/82; A01H 1/02

(52) U.S. Cl. CPC C12N 15/8281; C12N 15/8273; A01H 1/02

(57) ABSTRACT

The present disclosure provides methods for regulating stomata in plants, improving drought tolerance, and increasing resistance to bacterial pathogens through overexpression of genes NHR1 or GCN4. Also provided are transgenic plants with improved drought tolerance and increased resistance to bacterial pathogens produced by such methods.

### CROSS-REFERENCE TO RELATED APPLICATIONS

This application claims the benefit of U.S. Provisional Application No. 62/278,881, filed Jan. 14, 2016, herein incorporated by reference in its entirety.

### FIELD OF THE INVENTION

The present disclosure relates generally to the field of molecular biology. More specifically, the disclosure relates to genes involved in plant regulation, drought tolerance, resistance to bacterial pathogens, and methods of use thereof.

### INCORPORATION OF SEQUENCE LISTING

The sequence listing contained in filename “NBLE091US_ST25.txt” was created on Jan. 11, 2017, is 33 kilobytes as measured in Microsoft Windows operating system, and is filed electronically herewith and incorporated herein by reference.

### BACKGROUND OF THE INVENTION

Plants are constantly exposed to potential pathogens present in the environment. In response, plants have evolved intricate defense mechanisms. A common and durable plant defense mechanism is nonhost resistance. Nonhost resistance is achieved by a combination of preformed and inducible defenses. Stopping the entry of the pathogen into plant tissue is a key aspect of nonhost resistance. Bacterial pathogens rely on wounds or natural openings to enter the plant apoplast. One well-characterized means of entry is through the stomata, microscopic pores on the plant surface that allow gas exchange between the plant and the atmosphere. The opening and closing of stomata depends on the environmental and physiological conditions of the plant and is regulated by two guard cells that surround the pore. Plants can sense the presence of bacteria and close their stomata upon recognition of a pathogen. Stomatal closure also occurs in response to both abiotic and biotic signals that may share common steps in guard cell signaling.

Genetic modification of plants has, in combination with conventional breeding programs, led to significant increases in agricultural yield over the last decades. Genetic manipulation of genes regulating plant structures, such as stomata, can improve drought tolerance and increase resistance to bacterial pathogens thereby enhancing production of valuable commercial crops. Accordingly, methods capable of improving plant drought tolerance and increasing plant resistance to bacterial pathogens through gene regulation are described.

### SUMMARY OF THE INVENTION

In one aspect, the present disclosure provides a method of increasing drought tolerance and resistance to bacterial infection including overexpressing a NHR1 or GCN4 gene, or both, in a plant. In one embodiment, the drought tolerance and resistance to bacterial infection is increased as compared to a plant that lacks said overexpression. In particular embodiments, overexpressing of the NHR1 or GCN4 gene, or both, includes expression of an exogenous NHR1 or GCN4 gene, or both. In some embodiments, overexpressing of the NHR1 or GCN4 gene, or both, includes expression of an endogenous NHR1 or GCN4 gene, or both.

In another embodiment, the NHR1 gene is NHR1A or NHR1B. In yet another embodiment, the plant can be a monocotyledonous plant. In some embodiments, the monocotyledonous plant is selected from the group consisting of corn, rice, wheat, sorghum, barley, oat, switchgrass, and turfgrass. In other embodiments, the plant can be dicotyledonous plant. In yet other embodiments, the dicotyledonous plant is selected from the group consisting of is a cotton, soybean, rapeseed, sunflower, tobacco, sugarbeet, and alfalfa. In certain embodiments, the plant has altered morphology as compared to a plant that lacks said overexpression. In one embodiment, the altered morphology is reduced stomatal aperture.

In another aspect, the present disclosure provides a plant including overexpression of a NHR1 or GCN4 gene, or both. In one embodiment, the drought tolerance and resistance to bacterial infection is increased as compared to a plant that lacks said overexpression.

In yet another aspect, the present disclosure provides a seed that produces a plant including overexpression of a NHR1 or GCN4 gene, or both. In one embodiment, the drought tolerance and resistance to bacterial infection is increased as compared to a plant that lacks said overexpression.

In one aspect, the present disclosure provides a seed produced by a plant including overexpression of a NHR1 or GCN4 gene, or both. In certain embodiments, the drought tolerance and resistance to bacterial infection is increased as compared to a plant that lacks said overexpression.

In a particular aspect, the present disclosure provides a DNA-containing plant part of a plant including overexpression of a NHR1 or GCN4 gene, or both. In some embodiments, the drought tolerance and resistance to bacterial infection is increased as compared to a plant that lacks said overexpression. In another embodiment, the plant part can be further defined as a protoplast, cell, meristem, root, leaf, node, pistil, anther, flower, seed, embryo, stalk or petiole.

In a certain aspect, the present disclosure provides a method of producing a plant including increased drought tolerance and resistance to bacterial infection. In one embodiment, the method includes obtaining a plant including overexpression of a NHR1 or GCN4 gene, or both. In another embodiment, the drought tolerance and resistance to bacterial infection is increased as compared to a plant that lacks said overexpression. In yet another embodiment, the method includes growing said plant. In certain embodiments, the method includes crossing said plant with itself or another distinct plant to produce progeny plants. In particular embodiments, the method includes selecting a progeny plant including overexpression of a NHR1 or GCN4 gene, or both. In some embodiments, said progeny plant includes increased drought tolerance and resistance to bacterial infection as compared to a plant that lacks said overexpression.

In one aspect, the present disclosure provides a transgenic plant including a recombinant DNA molecule. In one embodiment, the recombinant DNA molecule overexpresses a NHR1 or GCN4 gene, or both. In another embodiment, said overexpression increases drought tolerance and resistance to bacterial infection. In yet another embodiment, the recombinant DNA molecule includes a heterologous promoter operably linked to an exogenous NHR1 or GCN4 gene, or both. In still another embodiment, the NHR1 gene is NHR1A or NHR1B. In other embodiments, the transgenic plant can be further defined as a legume. In some embodiments, the transgenic plant can be further defined as an R0 transgenic plant. In particular embodiments, the transgenic plant can be further defined as a progeny plant of any generation of an R0 transgenic plant. In certain embodiments, the transgenic plant can inherit the recombinant DNA molecule from the R0 transgenic plant.

### BRIEF DESCRIPTION OF THE DRAWINGS

FIG. 1A, FIG. 1B and FIG. 1C show N. benthamiana NbNHR1-silenced plants are compromised in nonhost resistance and elicitation of HR. Representative histograms are depicted of NbNHR1-silenced (TRV::NbNHR1) and non-silenced control (TRV::00) N. benthamiana plants that were vacuum-inoculated with nonhost pathogen P. syringae pv. tomato T1 (pDSK-GFPuv) (FIG. 1A) or the host pathogen P. syringae pv. tabaci (pDSK-GFPuv) (FIG. 1B) to observe bacterial multiplication three days post-inoculation (dpi). An increase in GFP fluorescence associated with bacterial multiplication was observed in NbNHR1-silenced plants but not in the non-silenced controls (TRV::00). Bars represent means and standard deviations for three independent experiments. Asterisks indicate a statistically significant difference between NbNHR1-silenced and control plants (Student's t-test; p-value=0.05). Down-regulation of NbNHR1 was quantified and NbActin used as an internal control (FIG. 1C).

FIG. 2A and FIG. 2B shows N. benthamiana NbNHR1-silenced plants are compromised in nonhost resistance against different nonhost pathogens such as P syringae pv. glycinea and X. campestris pv. vescatoria. Representative histograms are depicted of NbNHR1-silenced (TRV::NbNHR1) and non-silenced control (TRV::00) N. benthamiana plants. Plants were vacuum-infiltrated with P. syringae pv. glycinea (FIG. 2A) and Xi campestris pv. vesicatoria (FIG. 2B) and bacterial growth was monitored at 0, 4 and 7 dpi.

FIG. 3A, FIG. 3B and FIG. 3C show NHR-silenced tomato plants are compromised in nonhost resistance against P. syringae pv. tabaci (Pstab). Representative histograms are depicted of SlNHR1-silenced tomato plants sprayed with nonhost pathogen Pstab and host pathogen P. syringae pv. tomato DC3000 (Pst DC3K). Down-regulation of SlNHR1 was quantified, and SlActin used as an internal control (FIG. 3A). Bacterial growth was measured 2 and 6 dpi for Pst DC3K (FIG. 3B) and Pstab (FIG. 3C). Bars represent the means and standard deviation from three independent experiments. Asterisks indicate a statistically significant difference between treatments for equivalent time points (Student's t-test; p-value=0.05).

FIG. 4A and FIG. 4B show AtNHR1A and AtNHR1B are highly conserved among different organisms and have sequence similarity to the small GTP-binding family proteins Obg, DRG and ERG. An amino acid sequence alignment generated using PRALINE is depicted of AtNHR1A, AtNHR1B, and orthologous genes in N. benthamiana, tomato, yeast and human (FIG. 4A). Sequence similarities are represented by different colored boxes. The predicted domain for GTPase is marked by a black box. A neighbor-joining tree generated by Mega5 software (Tamura et al., 2011) depicts the phylogenetic analysis of AtNHR1A and AtNHR1B (FIG. 4B). Branch lengths are proportional to the estimated evolutionary distance. Numbers next to branches indicate bootstrap values.

FIG. 5 shows an amino acid sequence alignment of AtNHR1A and AtNHR1B among different Arabidopsis ecotypes, showing the early termination and truncation of AtNHR1A in four ecotypes, Col-0, Ler-0, Rsch-4 and Wil-2.

FIG. 6A, FIG. 6B and FIG. 6C show ABA, PAMPs, host and nonhost bacterial pathogens induce AtNHR1A, AtNHR1B, and stomatal closure in an AtNHR1A-dependent manner. Histograms are depicted of AtNHR1A (FIG. 6A) and AtNHR1B (FIG. 6B) gene expression in Arabidopsis wild-type (Col-0) plants individually syringe-infiltrated with ABA (10 μM), Flg22 (20 μM), or LPS (100 ng), or flood-inoculated with the pathogens P. syringae pv. maculicola (Psm) and P. syringae pv. tabaci (Pst) at 1×10⁴ cfu/mL. Bars indicate relative gene expression in comparison with the housekeeping gene UBQ5 and in relation to the 0 hr time-point. Different letters above bars indicate a statistically significant difference within a treatment (Student's t-test; p-value=0.05). Error bars represent the standard deviation of three biological replicates (three technical replicates for each biological replicate). Histograms are depicted quantifying stomatal aperture size from epidermal peels incubated with ABA (10 μM or 50 μM), Flg22 (20 μM), LPS (100 ng) and the nonhost pathogen Pst (1×10⁶ cfu/mL), where aperture size was measured after 30 min for ABA, 1 hr for flg22 and 3 hrs for Pst (FIG. 6C).

FIG. 7A and FIG. 7B show the different patterns of AtNHR1A and AtNHR1B expression in Arabidopsis mesophyll and guard cells. Representative histograms are depicted of AtNHR1A (FIG. 7A) and AtNHR1B (FIG. 7B) expression in mesophyll cells and guard cells after 4 hrs of treatment with 100 μM ABA.

FIG. 8A and FIG. 8B show the position of T-DNA insertions in AtNHR1A and expression of AtNHR1A in wild-type and mutants. A representative illustration is depicted of the NHR1A coding sequence (FIG. 8A). Exons are shown as black boxes and arrowheads indicate the position of primers used to examine NHR1A gene expression. A representative histogram is depicted of AtNRH1A expression in nhr1a and NHR1A-OE lines based on qRT-PCR results obtained using RNA from two-week old seedlings (FIG. 8B). AtActin2 and AtUBQ5 were used as internal controls from normalization.

FIG. 9A and FIG. 9B show bacterial entry through stomata in an nhr1a Arabidopsis mutant and NbNHR1-silenced N. benthamiana incubated with host and nonhost pathogens, P. syringae pv. maculicola (Psm; FIG. 9A) and P. syringae pv. tabaci (Pstab; FIG. 9B) expressing GFPuv (Wang et al., 2007), respectively. Bacterial entry through stomata was observed 2 hpi. Representative histograms depict bacterial entry 1 hpi and 3 hpi. Arrows indicate stomata in epidermal peels. Scale bars=10 μm.

FIG. 10A and FIG. 10B show down-regulation of AtNHR1B by RNAi and associated phenotypes. Representative histograms are depicted of AtNHR1B qRT-PCR results in various independent transgenic lines (FIG. 10A) and double mutant mimics, nhr1a NHR1B-RNAiA and nhr1a NHR1B-RNAiB (FIG. 10B). AtUBQ5 was used as an internal control.

FIG. 11A and FIG. 11B show AtNHR1B-RNAi lines are compromised in nonhost disease resistance. Representative histograms are depicted of Arabidopsis wild-type (Col-0), Atnhr1a mutant, AtNHR1B-RNAi, Atnhr1a AtNHR1B-RNAi double-mutant mimic, overexpression (AtNHR1A-OE and AtNHR1B-OE), and complementation lines (AtNHR1A-comp) that were flood-inoculated with the nonhost pathogen P. syringae pv. tabaci (FIG. 11A) or host pathogen P. syringae pv. maculicola (FIG. 11B) at 1×10⁴ cfu/mL to assess disease symptoms and bacterial growth 1 and 3 dpi. Different letters above bars indicate a statistically significant difference within a time point (Student's t-test; p-value=0.05). Bars represent the means and standard deviation of three biological replications (three technical replicates for each biological replication).

FIG. 12A, FIG. 12B and FIG. 12C show NHR1A has GTPase activity and interacts with JAZ9 in Arabidopsis. A representative histogram is depicted showing that nhr1a is less sensitive to JA than Col-0, where roots were measured 7 days after seeds of different Arabidopsis lines were grown in MS medium plates with or without 30 μM of MeJA (FIG. 12A). Data represents three independent experiments with at least 10 seedlings per line. Bars represent the mean±SD. Asterisks indicate statistical significance (Student's t-test; p-value <0.05). A representative scatter-plot and histogram are depicted showing that the GTPase activity of NHR1A is reduced by JAZ9 as measured by the rate of phosphate (Pi) release when NHR1A protein (1 μM) was pre-loaded with GTP (1 mM) and incubated without (FIG. 12B) or with 0.25-1 μM of JAZ9 (FIG. 12C). Data represents three independent experiments. Bars represent the mean±SE.

FIG. 13 shows reduction of NHR1A GTPase activity after binding with JAZ9. A representative scatter plot is depicted of GTP binding and GTP hydrolysis of NHR1A protein measured using GTP-BODIPY-FL in a real-time fluorescence assay in presence or absence of JAZ9 protein. Data represents one of two independent experiments each with three replicates. Points represent the mean±SE.

FIG. 14A, FIG. 14B and FIG. 14C show gene expression profiling in the mutant of NHR1A and JAZ9 in Arabidopsis. Venn diagrams are depicted illustrating the number of up and down-regulated genes overlapping between nhr1a and jaz9 without treatment (FIG. 14A). Representative microarray scans are depicted of the differential expression of guard cell signaling genes (FIG. 14B) and SA-, JA-, and PTI-mediated defense pathway marker genes (FIG. 14C) in Col-0 and nhr1a at various time-points after inoculation with ABA, COR, P. syringae pv. maculicola and P. syringae pv. tabaci. OST1: Open Stomata 1, rbohD: Respiratory Burst Oxidase Homologue D, MPK4: MAP Kinase, ABI1: ABA Insensitive 1, SLAC1: Slow Anion Channel-Associated 1, RIN4: Rpm1 Interaction Protein 4, SLAH3: SLAC1 Homologue 3, CPK4: Calcium-Dependent Protein Kinase 4, EDS1: Enhanced Disease Susceptibility 1, PR1: Pathogenesis-Related Gene 1, AOS: Allene Oxide Synthase, PDF1.2: Plant Defensin 1.2, LOX2: Lipoxygenase, FLS2: Flagellin Sensitive 2, BAK1: BRI 1-Associated Receptor Kinase 1, COI1: Coronatine Insensitive 1.

FIG. 15 shows functional involvement of AtNRH1A in JA and ABA hormonal signaling in response to abiotic stresses. A representative microarray dataset is depicted demonstrating the down-regulated genes in both nhr1a and jaz9 mutants compared to Col-0.

FIG. 16 shows a model of NHR1A function in stomata-mediated defense response to abiotic and biotic stimuli. COI1 recruits JAZ9 for ubiquitination and degradation in the presence of COR/JA. NHR1A interacts with JAZ9 for regulating JA-mediated stomata closure in response to bacterial pathogens but acts in a pathway independent of ABA. NHR1A can also be involved in MAP kinases-mediated ABA signaling pathway for stomatal open/closure. NHR1A localizes to nuclei like JAZ9 and MYC2. NHR1A can participate in the cross-talk between JAZ9 and MYC2 for regulating JA signal transduction pathway.

FIG. 17 shows gene expression of Nb4D7-2 in Nb4D7-2-silenced N. benthamiana plants (TRV::Nb4D7-2) and non-silenced controls (TRV:GFP) as determined by quantitative RT-PCR (qRT-PCR).

FIG. 18A and FIG. 18B show silencing of Nb4D7-2 in N. benthamiana enhances growth of the nonhost pathogen P. syringae pv. tomato T1 bacteria and confers hyper-susceptibility to the host pathogen P. syringae pv. tabaci. Representative histograms are depicted of wild-type, silenced (TRV:4D7-2), and non-silenced plants (TRV:GFP) that were vacuum inoculated with a GFPuv-expressing non-host pathogen, P. syringae pv. tomato T1 (pDSK-GFPuv) (FIG. 18A), and the host pathogen P. syringae pv. tabaci (pDSK-GFPuv) (FIG. 18B). Bars represent the mean and standard deviation (SD) for four biological replicates in three independent experiments.

FIG. 19A and FIG. 19B show Arabidopsis plants overexpressing GCN4 are resistant to the host pathogen P. syringae pv, maculicola. Representative histograms are depicted of wild-type Col-0 and GCN4 overexpressing lines (AtGCN4-OE6 and AtGCN4-OE16) that were flood-inoculated (FIG. 19A) or syringe-inoculated (FIG. 19B) with the host pathogen P. syringae pv maculicola with bacterial growth quantified at 0 and 3 dpi. Bars represent the mean and SD for four biological replicates in three independent experiments.

FIG. 20 shows AtGCN4 overexpressing Arabidopsis are unable to reopen stomata after treatment with the host pathogen P. syringae pv. tomato DC3000 and purified coronatine (COR). A representative histogram is depicted of stomatal aperture in Arabidopsis plant epidermal peels from wild-type Col-0 and GCN4-overexpressing (AtGCN4-OE6 and AtGCN4-OE16) lines measured 4 hours after treatment with MES buffer, COR, ABA, or COR+ABA.

FIG. 21 shows a representative scatter-plot of the rate of water loss estimated in Col-0 and AtGCN4-overexpressing plants (AtGCN4-OE6 and AtGCN4-OE16). Values are the mean±SE (n=6 plants; *p-value <0.05).

FIG. 22 shows a representative histogram of the stomatal aperture in NbGCN4-silenced N. benthamiana and non-silenced controls after inoculation with P. syringae pv tomato T1. Bars represent the mean and SD.

FIG. 23A, FIG. 23B, FIG. 23C and FIG. 23D show measurement of physiological parameters showing drought tolerance in NHR1A and NHR1B OX lines. FIG. 23A. Cell sap osmolality. FIG. 23B. Relative water content in the leaves. FIG. 23C. ABA levels. FIG. 23D. Leaf water loss.

### DETAILED DESCRIPTION OF THE INVENTION

The present disclosure provides a method of increasing drought tolerance and resistance to bacterial infection in a plant by increasing expression or overexpressing a NHR1 gene, a GCN4 gene, or both. Plants of the present disclosure that exhibit increased expression or overexpression of a NHR1 gene, a GCN4 gene, or both, demonstrate beneficial traits including increased drought tolerance and resistance to bacterial infection as compared to a plant that lacks said increased expression or overexpression.

The ability of plants to withstand bacterial infection and survive in water-poor conditions is controlled by a plant's genetic make-up. Plant lateral organs are primary sources of food and feed and as such, methods for increasing these would be beneficial. To facilitate an improvement in crop survival, the inventors provide for the first time a small GTP-binding protein NONHOST RESISTANCE (NHR) 1 (existing as two copies in all plant species, NHR1A and NHR1B), and an ABC transporter F family 4 protein, GCN4 (general control repressible-4). These genes are involved in the regulation of plant stomata and, thus, drought tolerance and resistance to bacterial infection. By increasing expression, or overexpressing, NHR1A, NHR1B, GCN4, or a combination thereof in plants using recombinant DNA molecules, the inventors have been able to significantly increase the drought tolerance and resistance to bacterial infection of plants, thereby providing a powerful strategy for increasing crop survivability.

In one embodiment, a plant in accordance with the disclosure having increased drought tolerance and resistance to bacterial infection can comprise increased expression of an endogenous NHR1 gene sequence, or GCN4 gene sequence, or both. In another embodiment, a plant with increased drought tolerance and resistance to bacterial infection can comprise overexpression of an exogenous NHR1 gene sequence, or GCN4 gene sequence, or both. In other embodiments, the disclosure provides primers which may be useful for detection or amplification of a sequence as described herein. Such sequences are set forth herein as SEQ ID NOs:3-6. In another embodiment, such primers may be useful for detecting the presence of absence of a gene or sequence of the disclosure. In accordance with the disclosure, nucleic acid and/or protein sequences may share sequence identity at the nucleic acid or amino acid level. For example, such sequences may share 100%, 99%, 98%, 97%, 96%, 95%, 94%, 93%, 92%, 91%, 90%, 89%, 88%, 87%, 86%, 85%, 84%, 83%, 82%, 81%, 80% sequence identity, or the like.

In some embodiments, a plant according to the disclosure may be a monocotyledonous plant or a dicotyledonous plant. In other embodiments, the plant may be a forage plant, a biofuel crop, a cereal crop, or an industrial plant. In one embodiment, a forage plant may include, but is not limited to, a forage soybean, alfalfa, clover, Bahia grass, Bermuda grass, dallisgrass, pangolagrass, big bluestem, indiangrass, fescue (Festuca sp.), Dactylis sp., Brachypodium distachyon, smooth bromegrass, orchardgrass, Kentucky bluegrass, reed canarygrass plant, switchgrass (Panicum virgatum), or the like. In certain other embodiments, the plant may be a biofuel crop including, but not limited to, switchgrass (Panicum virgatum), giant reed (Arundo donax), reed canarygrass (Phalaris arundinacea), Miscanthus×giganteus, Miscanthus sp., sericea lespedeza (Lespedeza cuneata), corn, sugarcane, sorghum, millet, ryegrass (Lolium multiflorum, Lolium sp.), timothy, Kochia (Kochia scoparia), soybeans, alfalfa, tomato, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, Kentucky bluegrass or poplar. Cereal crops for use according to the present disclosure include, but are not limited to, maize, rice, wheat, barley, sorghum, millet, oat, rye, triticle, buckwheat, fonio, and quinoa.

### I. Nucleic Acids, Polypeptides, and Plant Transformation Constructs

Certain embodiments of the current disclosure concern recombinant nucleic acid sequences comprising a NHR1A, NHR1B, or GCN4 coding sequence. The disclosure also provides sequences complementary to such sequences. Also provided are primers for detecting or amplifying a sequence in accordance with the disclosure, which are set forth herein as SEQ ID NOs:3-6. Complements to any nucleic acid sequences described herein are also provided.

“Identity,” as is well understood in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. Methods to determine “identity” are designed to give the largest match between the sequences tested. Moreover, methods to determine identity are codified in publicly available programs. “Identity” can be readily calculated by known methods including, but not limited to, those described in Lesk, ed., (1988); Smith, ed., (1993); Griffin, and Griffin, eds., (1994); von Heinje, (1987); Gribskov and Devereux, eds., (1991); and Carillo and Lipman, (1988). Computer programs that can be used to determine “identity” between two sequences may include but are in no way limited to, GCG (Devereux, 1984); suite of five BLAST programs, three designed for nucleotide sequences queries (BLASTN, BLASTX, and TBLASTX) and two designed for protein sequence queries (BLASTP and TBLASTN) (Coulson, 1994; Birren, et al., 1997). The BLASTX program is publicly available from NCBI and other sources (BLAST Manual, Altschul et al., NCBI NLM NIH, Bethesda, Md. 20894; Altschul et al., 1990). The well-known Smith Waterman algorithm can also be used to determine identity.

Parameters for polypeptide sequence comparison include the following: Algorithm: Needleman and Wunsch (1970); Comparison matrix: BLOSUM62 from Hentikoff and Hentikoff, (1992); Gap Penalty: 12; and Gap Length Penalty: 4. A program which can be used with these parameters is publicly available as the “gap” program from Genetics Computer Group, Madison Wis. The above parameters along with no penalty for end gap may serve as default parameters for peptide comparisons.

Parameters for nucleic acid sequence comparison are known in the art and may include the following: Algorithm: Needleman and Wunsch (1970); Comparison matrix: matches=+10; mismatches=0; Gap Penalty: 50; and Gap Length Penalty: 3. A program which can be used with these parameters is publicly available as the “gap” program from Genetics Computer Group, Madison Wis. The above parameters may serve as the default parameters for nucleic acid comparisons.

As used herein, “hybridization,” “hybridizes,” or “capable of hybridizing” is understood to mean the forming of a double- or triple-stranded molecule or a molecule with partial double- or triple-stranded nature. Such hybridization may take place under relatively high-stringency conditions, including low salt and/or high temperature conditions, such as provided by a wash in about 0.02 M to about 0.15 M NaCl at temperatures of about 50° C. to about 70° C. for 10 min. In one embodiment of the disclosure, the conditions are 0.15 M NaCl and 70° C. Stringent conditions tolerate little mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

The nucleic acids provided herein may be from any source, e.g., identified as naturally occurring in a plant, or synthesized, e.g., by mutagenesis of a sequence set forth herein. In an embodiment, the naturally occurring sequence may be from any plant. In certain embodiments, the plant can be a monocotyledonous plant or a dicotyledonous plant.

Coding sequences, such as a NHR1 coding sequence, or a GCN4 coding sequence, or complements thereof, may be provided in a recombinant vector or construct operably linked to a heterologous promoter functional in plants, in either sense or antisense orientation. In other embodiments, plants and plant cells transformed with the sequences may be provided. The construction of vectors which may be employed in conjunction with plant transformation techniques using these or other sequences according to the disclosure will be known to those of skill of the art in light of the present disclosure (e.g., Sambrook et al., 1989; Gelvin et al., 1990). The techniques of the current disclosure are thus not limited to any particular nucleic acid sequences.

The choice of any additional elements used in conjunction with the NHR1 or GCN4 sequences may depend on the purpose of the transformation. One of the major purposes of transformation of crop plants is to add commercially desirable, agronomically important traits to the plant, as described herein. Such traits may include, but are not limited to increased drought tolerance, increased resistance to bacterial infection, pesticide resistance, herbicide tolerance, increased seed yield, increased seed size and weight, increased pod size, increased leaf size, and increased plant biomass, and the like.

Vectors or constructs used for plant transformation may include, for example, plasmids, cosmids, YACs (yeast artificial chromosomes), BACs (bacterial artificial chromosomes) or any other suitable cloning system known in the art, as well as fragments of DNA therefrom. Thus, when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. In accordance with the disclosure, this could be used to introduce genes corresponding to, e.g., an entire biosynthetic pathway, into a plant.

Particularly useful for transformation are expression cassettes which have been isolated from such vectors. DNA segments used for transforming plant cells will generally comprise the cDNA, gene, or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant recombinant cells resulting in a screenable or selectable trait and/or which will impart an improved phenotype to the resulting transgenic plant. In an embodiment, introduction of such a construct into a plant may result in increased expression of a particular gene in the plant. In another embodiment, introduction of such a construct may result in reduction or elimination of expression of a particular gene. Preferred components likely to be included with vectors used in the current disclosure are as follows.

A. Regulatory Elements

As used herein, “increased expression” or “overexpression” can refer to any of the well-known methods for increasing the levels of protein produced as a result of gene transcription to mRNA and subsequent translation of the mRNA. Increased expression and overexpression also refer to the substantial and measurable increase in the amount of mRNA in the cell. The transcribed RNA can be in the sense orientation, in the anti-sense orientation, or in both orientations. Such expression may be effective against a endogenous, native plant gene associated with a trait, or an exogenous gene that may be introduced into the plant.

The use of recombinant DNA molecules for increasing expression of an endogenous gene or overexpressing an exogenous gene in plants is well known in the art. Exemplary promoters for expression of a nucleic acid sequence include plant promoters such as the CaMV 35S promoter (Odell et al., 1985), or others such as CaMV 19S (Lawton et al., 1987), nos (Ebert et al., 1987), Adh (Walker et al., 1987), sucrose synthase (Yang and Russell, 1990), α-tubulin, actin (Wang et al., 1992), cab (Sullivan et al., 1989), PEPCase (Hudspeth and Grula, 1989) or those promoters associated with the R gene complex (Chandler et al., 1989). Tissue-specific promoters such as leaf specific promoters, or tissue selective promoters (e.g., promoters that direct greater expression in leaf primordia than in other tissues), and tissue-specific enhancers (Fromm et al., 1986) are also contemplated to be useful, as are inducible promoters such as ABA- and turgor-inducible promoters. Any suitable promoters known in the art may be used to express a nucleic acid sequence in accordance with the disclosure in a plant. In one embodiment, such a nucleic acid sequence may encode a DNA sequence that results in increased expression or overexpression of a NHR1 gene, or a GCN4 gene, or both, in a plant. In a particular embodiment of the disclosure, the CaMV35S promoter or a native promoter may be used to express a nucleic acid sequence that results in increased expression or overexpression of a NHR1 gene, or a GCN4 gene, or both, in a plant.

The DNA sequence between the transcription initiation site and the start of the coding sequence, i.e., the untranslated leader sequence, can also influence gene expression. One may thus wish to employ a particular leader sequence with a transformation construct of the disclosure. In one embodiment, leader sequences are contemplated to include those which comprise sequences predicted to direct optimum expression of the attached gene, i.e., to include a consensus leader sequence which may increase or maintain mRNA stability and prevent inappropriate initiation of translation. The choice of such sequences will be known to those of skill in the art in light of the present disclosure. In some embodiments, sequences that are derived from genes that are highly expressed in plants may be used for expression of nucleic acid sequences targeting a NHR1 gene, a GCN4 gene, or both, in a plant.

It is envisioned that nucleic acid sequences targeting a NHR1 gene, or a GCN4 gene, or both, may be introduced under the control of novel promoters, enhancers, etc., or homologous or tissue-specific or tissue-selective promoters or control elements. Vectors for use in tissue-specific targeting of genes in transgenic plants will typically include tissue-specific or tissue-selective promoters and may also include other tissue-specific or tissue-selective control elements such as enhancer sequences. Promoters which direct specific or enhanced expression in certain plant tissues will be known to those of skill in the art in light of the present disclosure. These include, for example, the rbcS promoter, specific for green tissue; the ocs, nos and mas promoters, which have higher activity in roots.

B. Transcription Terminating Sequences

Transformation constructs prepared in accordance with the disclosure may include a 3′ end DNA sequence that acts as a signal to terminate transcription and allow for the polyadenylation of the mRNA produced by coding sequences operably linked to a promoter. In one embodiment of the disclosure, the native terminator of a NHR1 sequence, or a GCN4 sequence, or both, can be used. Alternatively, a heterologous 3′ end may enhance the expression of sense or antisense NHR1 sequences, GCN4 sequences, or both. Examples of such sequences that may be used in this context include those from the nopaline synthase gene of Agrobacterium tumefaciens (nos 3′ end) (Bevan et al., 1983), the terminator sequence for the T7 transcript from the octopine synthase gene of Agrobacterium tumefaciens, and the 3′ end of the protease inhibitor I or II gene from potato or tomato. Regulatory elements such as an Adh intron (Callis et al., 1987), sucrose synthase intron (Vasil et al., 1989) or TMV omega element (Gallie et al., 1989), may further be included where desired.

C. Transit or Signal Peptides

Sequences that are joined to the coding sequence of an expressed gene, which are removed post-translationally from the initial translation product and which facilitate the transport of the protein into or through intracellular or extracellular membranes, are termed transit (usually into vacuoles, vesicles, plastids and other intracellular organelles) and signal sequences (usually to the endoplasmic reticulum, Golgi apparatus, and outside of the cellular membrane). By facilitating the transport of the protein into compartments inside and outside the cell, these sequences may increase the accumulation of gene products by protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit or signal peptide will transport the protein to a particular intracellular or extracellular destination, respectively, and will then be post-translationally removed.

D. Marker Genes

By employing a selectable or screenable marker, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., β-glucuronidase (GUS), green fluorescent protein (GFP), or yellow fluorescent protein (YFP)). Of course, many examples of suitable marker proteins are known to the art and can be employed in the practice of the disclosure.

Many selectable marker coding regions are known and could be used with the present disclosure including, but not limited to, neo (Potrykus et al., 1985), which provides kanamycin resistance and can be selected for using kanamycin, G418, paromomycin, etc.; bar, which confers bialaphos or phosphinothricin resistance; a mutant EPSP synthase protein (Hinchee et al., 1988) conferring glyphosate resistance; a nitrilase such as bxn from Klebsiella ozaenae which confers resistance to bromoxynil (Stalker et al., 1988); a mutant acetolactate synthase (ALS) which confers resistance to imidazolinone, sulfonylurea or other ALS inhibiting chemicals (European Patent Application 154, 204, 1985); a methotrexate resistant DHFR (Thillet et al., 1988), a dalapon dehalogenase that confers resistance to the herbicide dalapon; or a mutated anthranilate synthase that confers resistance to 5-methyl tryptophan.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, (Murakami et al., 1986; Twell et al., 1989) causing rapid accumulation of ammonia and cell death.

One beneficial use of the sequences provided by the disclosure may be in the alteration of plant phenotypes by genetic transformation with nucleic acid molecules encoding NHR1 sequences, GCN4 sequences, or both. Such nucleic acid molecules may be provided with other sequences. Where an expressible coding region that is not necessarily a marker coding region is employed in combination with a marker coding region, one may employ the separate coding regions on either the same or different DNA segments for transformation. In the latter case, the different vectors are delivered concurrently to recipient cells to maximize co-transformation.

### II. Genetic Transformation

Additionally provided herein are transgenic plants transformed with a recombinant vector as described herein encoding or producing a NHR1 sequence, a GCN4 sequence, or both, or a sequence modulating expression thereof. In one embodiment, the disclosure provides a transgenic plant or plant cell comprising a polynucleotide molecule or a recombinant DNA construct as described herein, wherein the polynucleotide molecule or recombinant DNA construct encodes or produces a NHR1 sequence, a GCN4 sequence, or both, or a variant or homologue thereof. In a certain embodiment, the polynucleotide molecule or a recombinant DNA construct may result in the increased expression or overexpression of NHR1, GCN4, or both, in the plant. The disclosure therefore also provides progeny of these plants, vegetative, propagative, and reproductive parts of the plants comprising a transgene encoding a NHR1 sequence, a GCN4 sequence, or both. In some embodiments, a plant in accordance with the present disclosure comprises increased drought tolerance and resistance to bacterial infection relative to a plant not comprising such a polynucleotide molecule or DNA construct.

Suitable methods for transformation of plant or other cells for use with the current disclosure are believed to include virtually any method by which DNA can be introduced into a cell, such as by direct delivery of DNA, by Agrobacterium-mediated transformation (U.S. Pat. No. 5,591,616 and U.S. Pat. No. 5,563,055; both specifically incorporated herein by reference) by acceleration of DNA coated particles (U.S. Pat. No. 5,550,318; U.S. Pat. No. 5,538,877; and U.S. Pat. No. 5,538,880; each specifically incorporated herein by reference in its entirety), etc. Through the application of techniques such as these, the cells of virtually any plant species may be stably transformed, and these cells developed into transgenic plants.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (1985), Rogers et al., (1987) and U.S. Pat. No. 5,563,055, specifically incorporated herein by reference in its entirety.

Agrobacterium-mediated transformation is most efficient in dicotyledonous plants and is the preferable method for transformation of dicots, including Arabidopsis, tobacco, tomato, alfalfa and potato. Indeed, while Agrobacterium-mediated transformation has been routinely used with dicotyledonous plants for a number of years, including alfalfa (Thomas et al., 1990), it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., 1997; U.S. Pat. No. 5,591,616, specifically incorporated herein by reference in its entirety), wheat (McCormac et al., 1998), barley (Tingay et al., 1997; McCormac et al., 1998) and maize (Ishidia et al., 1996).

Modern Agrobacterium transformation vectors are capable of replication in E. coli as well as Agrobacterium, allowing for convenient manipulations as described (Klee et al., 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described (Rogers et al., 1987) have convenient multi-linker regions flanked by a promoter and a polyadenylation site for direct expression of inserted polypeptide coding genes and are suitable for present purposes. Gateway™ and other recombination-based cloning technology is also available in vectors useful for plant transformation. In addition, Agrobacterium containing both armed and disarmed Ti genes can be used for the transformations. In those plant strains where Agrobacterium-mediated transformation is efficient, it is the method of choice because of the facile and defined nature of the gene transfer.

One also may employ protoplasts for electroporation transformation of plants (Bates, 1994; Lazzeri, 1995). For example, the generation of transgenic soybean plants by electroporation of cotyledon-derived protoplasts is described by Dhir and Widholm in Intl. Patent Appl. Publ. No. WO 92/17598 (specifically incorporated herein by reference). Other examples of species for which protoplast transformation has been described include barley (Lazerri, 1995), sorghum (Battraw et al., 1991), maize (Bhattacharjee et al., 1997), wheat (He et al., 1994) and tomato (Tsukada, 1989).

Another method for delivering transforming DNA segments to plant cells in accordance with the disclosure is microprojectile bombardment (U.S. Pat. No. 5,550,318; U.S. Pat. No. 5,538,880; U.S. Pat. No. 5,610,042; and Intl. Patent Appl. Publ. No. WO 94/09699; each of which is specifically incorporated herein by reference in its entirety). In this method, particles may be coated with nucleic acids and delivered into cells by a propelling force. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species for which have been transformed by microprojectile bombardment include monocot species such as maize (Intl. Patent Appl. Publ. No. WO 95/06128), barley (Ritala et al., 1994; Hensgens et al., 1993), wheat (U.S. Pat. No. 5,563,055, specifically incorporated herein by reference in its entirety), rice (Hensgens et al., 1993), oat (Torbet et al., 1995; Torbet et al., 1998), rye (Hensgens et al., 1993), sugarcane (Bower et al., 1992), and sorghum (Casa et al., 1993; Hagio et al., 1991); as well as a number of dicots including tobacco (Tomes et al., 1990; Buising and Benbow, 1994), soybean (U.S. Pat. No. 5,322,783, specifically incorporated herein by reference in its entirety), sunflower (Knittel et al. 1994), peanut (Singsit et al., 1997), cotton (McCabe and Martinell, 1993), tomato (VanEck et al. 1995), and legumes in general (U.S. Pat. No. 5,563,055, specifically incorporated herein by reference in its entirety).

The transgenic plants of the present disclosure comprising increased expression or overexpression of NHR1, GCN4, or both can be of any species. In some embodiments, the transgenic plant is a dicotyledonous plant, for example an agronomically important plant such as soybean, Medicago truncatula, a poplar, a willow, a eucalyptus, a hemp, a Medicago sp., a Lotus sp., a Trifolium sp., a Melilotus sp., a Vinca sp., a Nicotiana sp., a Vitis sp., a Ricinus sp., or an Arabidopsis species. The plant can be an R₀ transgenic plant (i.e., a plant derived from the original transformed tissue). The plant can also be a progeny plant of any generation of an R₀ transgenic plant, wherein the transgenic plant has the nucleic acid sequence from the R₀ transgenic plant.

Seeds of the any above-described transgenic plants may also be provided, particularly where the seed comprises the nucleic acid sequence. Additionally contemplated are host cells transformed with the above-identified recombinant vector. In some embodiments, the host cell is a plant cell.

Also contemplated herein is a plant genetically engineered to exhibit increased expression, or overexpression, or a NHR1 gene, or GCN4 gene, or both, wherein the protein product (i.e., polypeptide) alters plant morphology. In certain embodiments, the altered plant morphology may be increased drought tolerance and resistance to bacterial infection. Such plants are described in the Examples, and may be useful, e.g., as commercial plants, due to their increased survivability.

The plants of these embodiments having increased expression or overexpression of NHR1, GCN4, or both, can be of any species. The species may be any monocotyledonous or dicotyledonous plant, such as those described herein. One of skill in the art will recognize that the present disclosure may be applied to plants of other species by employing methods described herein and others known in the art.

Application of these systems to different plant strains depends upon the ability to regenerate that particular plant strain from protoplasts. Illustrative methods for the regeneration of cereals from protoplasts have been described (Toriyama et al., 1986; Yamada et al., 1986; Abdullah et al., 1986; Omirulleh et al., 1993 and U.S. Pat. No. 5,508,184; each specifically incorporated herein by reference in its entirety). Examples of the use of direct uptake transformation of cereal protoplasts include transformation of rice (Ghosh-Biswas et al., 1994), sorghum (Battraw and Hall, 1991), barley (Lazerri, 1995), oat (Zheng and Edwards, 1990) and maize (Omirulleh et al., 1993).

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. A medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. The rate of cell growth also will vary among cultures initiated with the array of media that permit growth of that cell type.

Tissue that can be grown in a culture includes meristem cells, Type I, Type II, and Type III callus, immature embryos and gametic cells such as microspores, pollen, sperm, and egg cells. Type I, Type II, and Type III callus may be initiated from tissue sources including, but not limited to, immature embryos, seedling apical meristems, root, leaf, microspores and the like. Those cells which are capable of proliferating as callus also are recipient cells for genetic transformation.

Somatic cells are of various types. Embryogenic cells are one example of somatic cells which may be induced to regenerate a plant through embryo formation. Non-embryogenic cells are those which typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques which can be employed to select target cells may include, e.g., assessing cell morphology and differentiation, or may use various physical or biological means. Cryopreservation also is a possible method of selecting for recipient cells.

### III. Production and Characterization of Stably Transformed Plants

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the disclosure. In this case, one would then generally assay the potentially transformed cell population by exposing the cells to a selective agent or agents, or one would screen the cells for the desired marker gene trait.

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce, into the host cell, a marker gene which confers resistance to some normally inhibitory agent, such as an antibiotic or herbicide. Examples of antibiotics which may be used include the aminoglycoside antibiotics neomycin, kanamycin and paromomycin, or the antibiotic hygromycin. Resistance to the aminoglycoside antibiotics is conferred by aminoglycoside phosphotransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide which constitutes a desirable selection agent is the broad-spectrum herbicide bialaphos. Another example of a herbicide which is useful for selection of transformed cell lines in the practice of the disclosure is the broad-spectrum herbicide glyphosate. Glyphosate inhibits the action of the enzyme EPSPS which is active in the aromatic amino acid biosynthetic pathway. Inhibition of this enzyme leads to starvation for the amino acids phenylalanine, tyrosine, and tryptophan and secondary metabolites derived therefrom. U.S. Pat. No. 4,535,060 describes the isolation of EPSPS mutations which confer glyphosate resistance on the EPSPS of Salmonella typhimurium, encoded by the gene aroA. The EPSPS gene from Zea mays was cloned and mutations similar to those found in a glyphosate resistant aroA gene were introduced in vitro. Mutant genes encoding glyphosate resistant EPSPS enzymes are described in, for example, Intl. Patent Appl. Publ. No. WO 97/4103.

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 weeks, then transferred to media conducive to maturation of embryoids. Cultures are transferred every 2 weeks on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soilless plant growth mix, and hardened, e.g., in an environmentally controlled chamber, for example, at about 85% relative humidity, 600 ppm CO₂, and 25-250 microeinsteins m⁻² s⁻¹ of light. Plants may be matured in a growth chamber or greenhouse. Plants can be regenerated in from about 6 weeks to 10 months after a transformant is identified, depending on the initial tissue. During regeneration, cells are grown on solid media in tissue culture vessels. Illustrative embodiments of such vessels are Petri dishes and Plant Cons. Regenerating plants can be grown at about 19 to 28° C. After the regenerating plants have reached the stage of shoot and root development, they may be transferred to a greenhouse for further growth and testing.

To confirm the presence of the exogenous DNA or “transgene(s)” in the regenerating plants, a variety of assays may be performed. Such assays include, for example, “molecular biological” assays, such as Southern and Northern blotting and polymerase chain reaction (PCR); “biochemical” assays, such as detecting the presence of a protein product, e.g., by immunological means (ELISAs and western blots) or by enzymatic function; plant part assays, such as leaf or root assays; and also, by analyzing the phenotype of the whole regenerated plant.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR, e.g., the presence of a gene, but also demonstrates integration into the genome and characterizes each individual transformant.

Both PCR and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR techniques also may be used for detection and quantitation of RNA produced from introduced genes. In this application of PCR it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot northern hybridizations. These techniques are modifications of northern blotting and will only demonstrate the presence or absence of an RNA species.

The expression of a gene product is often determined by evaluating the phenotypic results of its expression. These assays also may take many forms including but not limited to analyzing changes in the chemical composition, morphology, or physiological properties of the plant. Chemical composition may be altered, for instance, by expression of genes encoding enzymes or storage proteins which change amino acid composition and may be detected by amino acid analysis, or by enzymes that change starch quantity which may be analyzed by near infrared reflectance spectrometry. Morphological changes may include, for instance, larger seeds, larger seed pods, larger leaves, greater stature, thicker stalks, and altered leaf-stem ratio, among others. Most often changes in response of plants or plant parts to imposed treatments are evaluated under carefully controlled conditions termed bioassays.

### IV. Evaluation of Increased Drought Tolerance and Resistance to Bacterial Infection

A plant useful for the present disclosure may be an R₀ transgenic plant. Alternatively, the plant may be a progeny plant of any generation of an R₀ transgenic plant, where the transgenic plant has the nucleic acid sequence from the R₀ transgenic plant.

Plants in accordance with the disclosure exhibiting increased expression or overexpression of NHR1, GCN4, or both, can also be used to produce crop plants with increased drought tolerance and resistance to bacterial infection, for example by obtaining the above-identified plant comprising increased expression or overexpression of NHR1, GCN4, or both, and growing said plant under plant growth conditions to produce plant tissue from the plant. The increased drought tolerance and resistance to bacterial infection can be subsequently used for any purpose, for example for improved survivability of food or commodity plant products.

### V. Breeding Plants of the Disclosure

In addition to direct transformation of a particular plant genotype with a construct prepared according to the current disclosure, transgenic plants may be made by crossing a plant having a recombinant DNA molecule of the disclosure to a second plant lacking the construct. For example, a recombinant nucleic acid sequence producing a NHR1 coding sequence, a GCN4 coding sequence, or both, can be introduced into a particular plant variety by crossing, without the need for ever directly transforming a plant of that given variety. Therefore, the current disclosure not only encompasses a plant directly transformed or regenerated from cells which have been transformed in accordance with the current disclosure, but also the progeny of such plants. As used herein, the term “progeny” denotes the offspring of any generation of a parent plant prepared in accordance with the instant disclosure, wherein the progeny comprises a selected DNA construct prepared in accordance with the disclosure. “Crossing” a plant to provide a plant line having one or more added transgenes relative to a starting plant line, as disclosed herein, is defined as the techniques that result in a transgene of the disclosure being introduced into a plant line by crossing a plant of a starting line with a plant of a donor plant line that comprises a transgene of the disclosure. To achieve this one could, for example, perform the following steps:
(a) plant seeds of the first (starting line) and second (donor plant line that comprises a transgene of the disclosure) parent plants;
(b) grow the seeds of the first and second parent plants into plants that bear flowers;
(c) pollinate a flower from the first parent plant with pollen from the second parent plant; and
(d) harvest seeds produced on the parent plant bearing the fertilized flower.

Backcrossing is herein defined as the process including the steps of:
(a) crossing a plant of a first genotype containing a desired gene, DNA sequence or element to a plant of a second genotype lacking the desired gene, DNA sequence or element;
(b) selecting one or more progeny plant containing the desired gene, DNA sequence or element;
(c) crossing the progeny plant to a plant of the second genotype; and
(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### VI. Definitions

Expression: The combination of intracellular processes, including transcription and translation, undergone by a coding DNA molecule such as a structural gene to produce a polypeptide. A plant in accordance with the disclosure may exhibit altered expression of a gene set forth herein. Such altered expression may include increased expression, decreased expression, or complete absence of expression.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence which is not normally present in a given host genome in the genetic context in which the sequence is currently found. In this respect, the sequence may be native to the host genome, but be rearranged with respect to other genetic sequences within the host sequence. The sequence may also be altered, i.e., mutated, with respect to the native regulatory sequence. For example, a regulatory sequence may be heterologous in that it is linked to a different coding sequence relative to the native regulatory sequence.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R₀ transgenic plant or may be from a progeny of any generation thereof that inherits a given transgenic sequence from a starting transgenic parent plant.

Promoter: A recognition site on a DNA sequence or group of DNA sequences that provides an expression control element for a structural gene and to which RNA polymerase specifically binds and initiates RNA synthesis (transcription) of that gene.

R₀ transgenic plant: A plant that has been genetically transformed or has been regenerated from a plant cell or cells that have been genetically transformed.

Regeneration: The process of growing a plant from a plant cell (e.g., plant protoplast, callus or explant).

Recombinant DNA molecule: A synthetic nucleic acid sequence including at least one genetic element which can be introduced, or has introduced, into a plant genome by genetic transformation.

Transformation construct: A chimeric DNA molecule which is designed for introduction into a host genome by genetic transformation. Preferred transformation constructs will comprise all of the genetic elements necessary to direct the expression of one or more exogenous genes. In particular embodiments of the instant disclosure, it may be desirable to introduce a transformation construct into a host cell in the form of an expression cassette.

Transformed cell: A cell in which the DNA complement has been altered by the introduction of an exogenous DNA molecule into that cell.

Transgene: A segment of DNA which has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences which are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

Although this specification discloses advantages in the context of certain illustrative, non-limiting embodiments, various changes, substitutions, permutations, and alterations may be made without departing from the scope of the appended claims. Further, any feature described in connection with any one embodiment may also be applicable to any other embodiment.

### EXAMPLES

The following examples are included to demonstrate preferred embodiments of the disclosure. It should be appreciated by those of skill in the art that the techniques disclosed in the examples which follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### Example 1

### Plant Growth, Pathogen Inoculation, and Bacterial Growth Assays.

N. benthamiana and tomato plants were grown in a greenhouse. Silenced and control N. benthamiana plants were inoculated with appropriate bacterial pathogens. Bacterial strains were grown at 28° C. for 24 hrs on KB medium containing the following antibiotics: rifampicin (50 μg/mL), kanamycin (25 μg/mL), chloramphenicol (25 μg/mL), and spectinomycin (25 μg/mL). To prepare bacterial inocula, culture media was centrifuged at 5000 rpm for 10 min and resuspended in water for bacterial growth assays using vacuum infiltration and spraying. Inoculated plants were then incubated in growth chambers at 90 to 100% RH for the first 24 hrs.

Arabidopsis thaliana mutants: SALK_043706 and SALK_072852 containing insertions in AtNHR1A were obtained from the Salk Institute Genomic Analysis Laboratory. Wild-type Col-0 and mutant plants were grown on ½ MS plates in a growth chamber at 21° C. with a 14 hrs photoperiod and a light intensity of about 100 μE m⁻² sec⁻¹. Four-week old plants were inoculated with appropriate host or nonhost bacterial pathogens, and bacterial growth was measured. For the bacterial growth assays in N. benthamiana and tomato, samples from inoculated leaves were collected at specific time points after inoculation by using a 0.5 cm leaf puncher. Leaf tissues were ground in sterile water, serially diluted and plated on KB plates supplemented with appropriate antibiotics. For the bacterial growth assays in Arabidopsis after flood-inoculation, inoculated leaves were surface-sterilized with 15% H₂O₂ for 3 min to eliminate epiphytic bacteria and then washed with sterile distilled water. The leaves were then homogenized in sterile distilled water, and serial dilutions were plated onto KB medium containing antibiotics. Bacterial growth was evaluated in three independent experiments.

### Example 2

### Transgenic Line Development.

To complement the nhr1a mutant, the full-length coding region of NRH1A was cloned into pMDC162, controlled by the NHR1A native promoter. This construct was transformed to GV3101, and transferred into the nhr1a mutant using Arabidopsis floral dip transformation (Bent, 2006). To knock-down NHR1B in Col-0, a partial sequence of NHR1B (˜400 bp) was selected using the pssRNAit program (Noble Foundation). This fragment was cloned into an RNAi vector (Invitrogen, NY) and transformed using Arabidopsis floral dip transformation. To make double-mutant mimics of NHR1A and NHR1B, an NHR1B-RNAi construct was transformed into nhr1a mutants. To examine the localization of JAZ9 and NHR1A, the full-length coding region of both genes was cloned into either pMDC45 or pMDC83.

### Example 3

### Yeast Two-Hybrid Analysis.

The full-length (1-360 aa from Col-0) and truncated versions (1-150 aa) were initially cloned into pDONR207 (Life Technologies, Inc., Carlsbad, Calif.) and subsequently transferred to the yeast two-hybrid bait vector pDEST32 (Life Technologies, Inc., Carlsbad, Calif.). To examine interactions between fusion proteins, both bait (AtNHR1A) and prey plasmids (Arabidopsis cDNA library) were co-transformed into a MaV203 yeast strain carrying three GAL4-inducible reporter genes (lacZ, HISS, and URA3). Bait-prey interactions were selected on synthetic dropout media lacking Leu and Trp (SC-Leu-Trp). Yeast colonies grown in SC-Leu-Trp were streaked on the medium lacking Leu, Trp, His, and Ura supplemented with 10 mM 3-AT (3-amino-1,2,4-triazole) with X-gal (20 μg/mL). Plasmids pEXP32/Krev1, pEXP22/RalGDS-m1, and pEXP22/RalGDS-m2 (Invitrogen, NY) were included as positive and negative controls for interaction. Clones containing only prey were tested for auto-activation by growing them on SC-Leu-His with 10 mM 3-AT. For β-galactosidase assays, yeast transformants were grown at 30° C. to mid-log phase (OD₆₆₀=0.5-1.0) in YPD liquid medium. The exact OD₆₆₀ of each culture was measured and then assayed for β-galactosidase activity using a yeast β-galactosidase assay kit (Pierce Biotechnology, Inc.). Activity of β-galactosidase was measured at OD₄₂₀ and calculated using the equation: β-galactosidase activity=1,000×OD₄₂₀/T×V×OD₆₆₀, in which T is reaction time (min) of incubation and V is volume of cells (mL) used in the assay.

### Example 4

### Analysis of Biomolecular Fluorescence Complementation (BiFC).

Target genes were cloned as a protein fusion to the N- or C-terminal half of yellow fluorescent protein (YFP). The full-length coding regions of genes were fused in-frame with the fragments corresponding to the N- (n-EYFP1-155) and C- (c-EYFP156-239) termini of YFP in 2×35S. BiFC expression constructs pSITE-n-EYFP-target gene and pSITE-n-EYFP-target gene were transformed into Agrobacterium strain GV2260 or GV3101, and co-infiltrated into N. benthamiana leaves or flood-inoculated in Arabidopsis. To examine false positive interactions, each construct alone was infiltrated. Four days after treatments, fluorescent images were observed with a confocal laser microscope (BioRad, CA).

### Example 5

RNA Extraction and Quantitative Real-Time PCR (qRT-PCR).

Total RNA was purified from Arabidopsis leaves infiltrated with water (mock control), nonhost pathogen P. syringae pv. tabaci (Psta), or host pathogen P. syringae pv. maculicola (Psm). Total RNA was extracted using TRIzol (Invitrogen, NT) and 2 treated or inoculated leaves were pooled to represent one biological replicate. Total RNA was treated with DNase I (Invitrogen, NY), and 1 μg RNA was used to generate cDNA using Superscript III reverse transcriptase (Invitrogen, NY) and oligo d(T)15-20 primers. The cDNA (1:20) was then used for qRT-PCR using Power SYBR Green PCR master mix (Applied Biosystems, Foster City, Calif., USA) with an ABI Prism 7900 HT sequence detection system (Applied Biosystems). Primers specific for AtUBQ5 were used to normalize small differences in template amounts. Average Cycle Threshold (CT) values calculated using Sequence Detection Systems (version 2.2.2; Applied Biosystems) from duplicate samples were used to determine the fold expression relative to controls.

### Example 6

### Histochemical and Fluorescent Microscopy Analyses.

To determine the expression patterns of AtNHR1A and AtNHR1B, the promoters of AtNHR1A (1.2 kb) and AtNHR1B (0.9 kb) were fused to a GUS reporter gene. AtNHR1A::GUS and NHR1B::GUS transgenic seedlings were incubated with GUS staining solution at 37° C. Staining was discarded and chlorophyll cleared by washing with 70% ethanol and keeping the leaves in ethanol for 72 hrs. GUS activity was analyzed by bright-field transmitted light microscopy, and images were taken by digital camera (Nikon). Confocal analysis of GFP expression was performed using a confocal microscope (BioRad, CA).

### Example 7

NHR1 Silencing Impairs Nonhost Resistance in Nicotiana benthamiana and Tomato Against Bacterial Pathogens, and Delays the Elicitation of Hypersensitive Response.

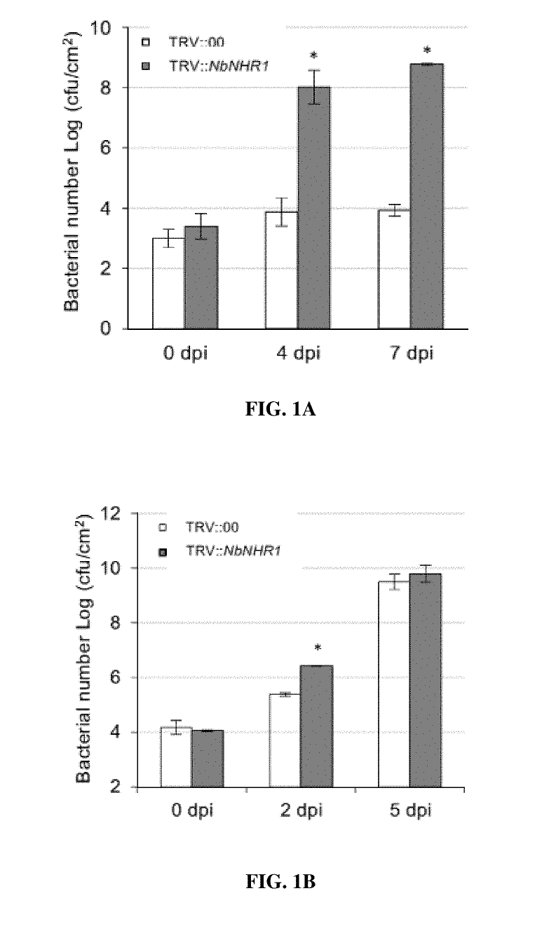

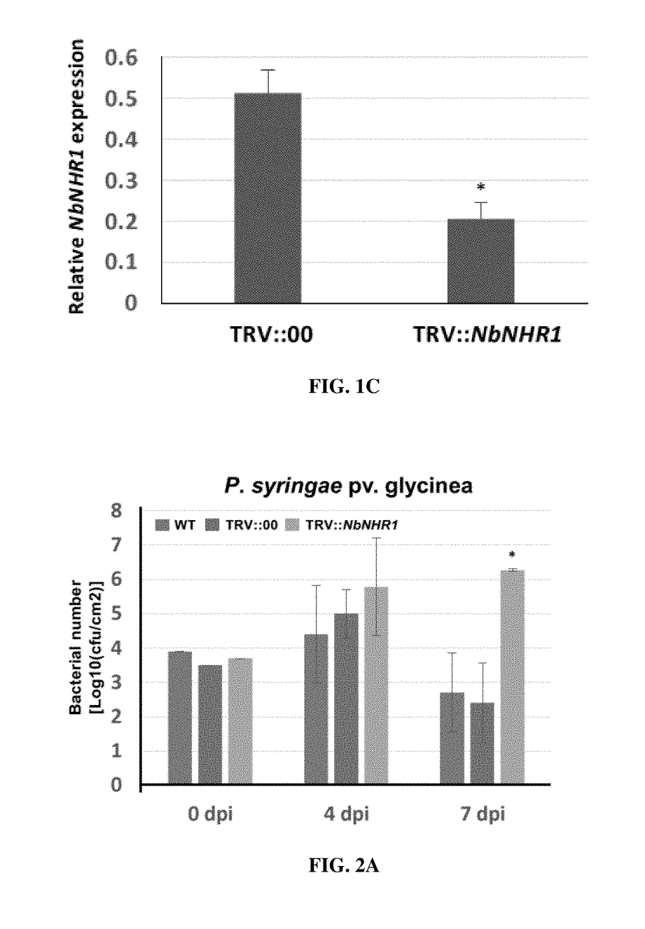

A Tobacco rattle virus (TRV)-based virus-induced gene silencing (VIGS)-mediated fast forward genetics approach was used in N. benthamiana to identify plant genes involved in nonhost resistance against bacterial pathogens (Wang et al., 2012). One of the identified cDNA clones had homology to an uncharacterized gene with a GTPase domain. This gene was named NONHOST RESISTANCE 1 (NHR1). Upon inoculation with the nonhost pathogen Pseudomonas syringae pv. tomato T1, that causes bacterial speck disease in tomato but not in the wild-type N. benthamiana, NHR1-silenced N. benthamiana plants showed disease symptoms characterized by chlorotic spots and significantly increased (>4 logs) bacterial multiplication in the inoculated leaves when compared to the non-silenced control (TRV::00) that was asymptomatic. Down-regulation of NbNHR1 was quantified and NbActin used as an internal control (FIG. 1C).

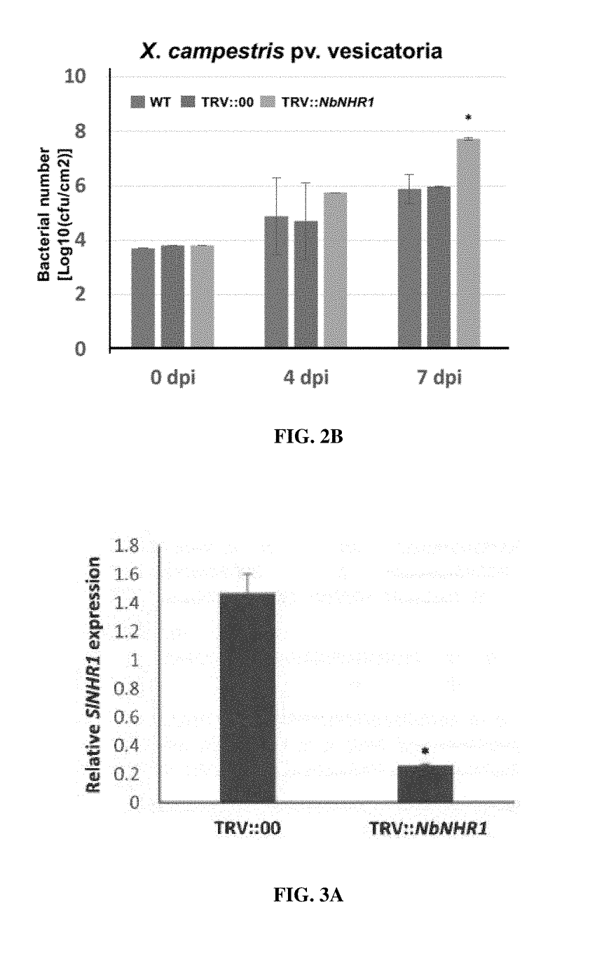

NbNHR1-silenced N. benthamiana plants were further analyzed to see if they were compromised in nonhost resistance against other nonhost pathogens such as P. syringae pv. glycinea (a bean pathogen) and Xanthomonas campestris pv. vesicatoria (a pepper pathogen). Both pathogens multiplied to significantly higher levels (100- to 1,000-fold) at seven days post-inoculation (dpi) in NHR1-silenced plants compared to wild-type and TRV::00 plants (FIG. 2A and FIG. 2B). Inoculation with the host pathogen P. syringae pv. tabaci caused disease symptoms and significant bacterial multiplication in both NbNHR1-silenced plants and non-silenced controls (TRV::00) with no significant difference at 5 dpi (FIG. 1B). To monitor bacterial multiplication in NbNHR1-silenced and non-silenced control (TRV:: 00) N. benthamiana plants were vacuum-infiltrated with P. syringae pv. tomato T1 (FIG. 1A) and P. syringae pv. tabaci (FIG. 1B), and bacterial multiplication was quantified at 0, 4 and 7 dpi for P. syringae pv. tomato T1 or 0, 2 and 5 dpi for P. syringae pv. tabaci.

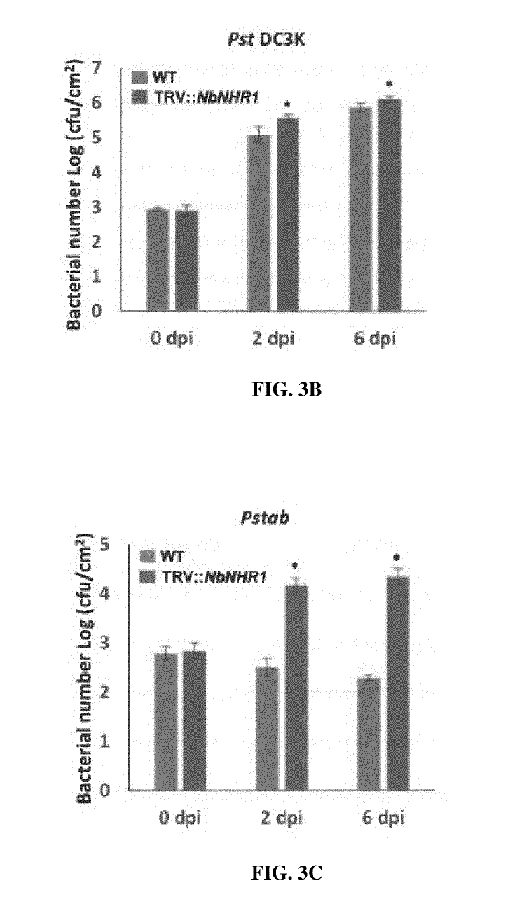

To determine if NHR1 was involved in nonhost disease resistance in more than one plant species, a N. benthamiana NHR1 gene was used to silence its orthologous gene in tomato (SlNHR1) using VIGS. Down-regulation of SlNHR1 was quantified, and SlActin used as an internal control (FIG. 3A). NHR1-silenced tomato plants and non-silenced control (TRV::00) were inoculated with the tomato nonhost pathogen P. syringae pv. tabaci that causes fire blight disease in tobacco. Similar to the findings in N. benthamiana, downregulation of SlNHR1 compromised nonhost disease resistance in tomato by producing disease symptoms and increased bacterial multiplication when compared to the control (FIG. 3C). Inoculation with the host pathogen P. syringae pv. tomato DC3000 caused slightly more disease symptoms accompanied with a higher bacterial titer in the SlNHR1-silenced plants than in TRV::00 plants (FIG. 3B). These results indicate that NHR1 is required for nonhost resistance against bacterial pathogens in N. benthamiana and tomato.

To determine if downregulation of NHR1 impairs elicitation of the hypersensitive response (HR), the onset of the HR was examined in NbNHR1-silenced and control plants after infiltration with high inoculum of the nonhost pathogens P. syringae pv. tomato T1 and X. campestris pv. vesicatoria, or by transient co-expression of the resistance (R) genes Pto or Cf9 with their corresponding avirulence genes AvrPto or AvrCf9, respectively, or by transient expression of the PAMP elicitor INF1. HR was observed in the control plants but not in the NbNHR1-silenced plants, indicating that NHR1 also plays a role in elicitation of the HR triggered by nonhost pathogens, gene-for-gene interactions and PAMPs.

### Example 8

### AtNHR1A and AtNHR1B are Members of the Small GTP-Binding Family Proteins Obg, DRG and ERG in Arabidopsis.

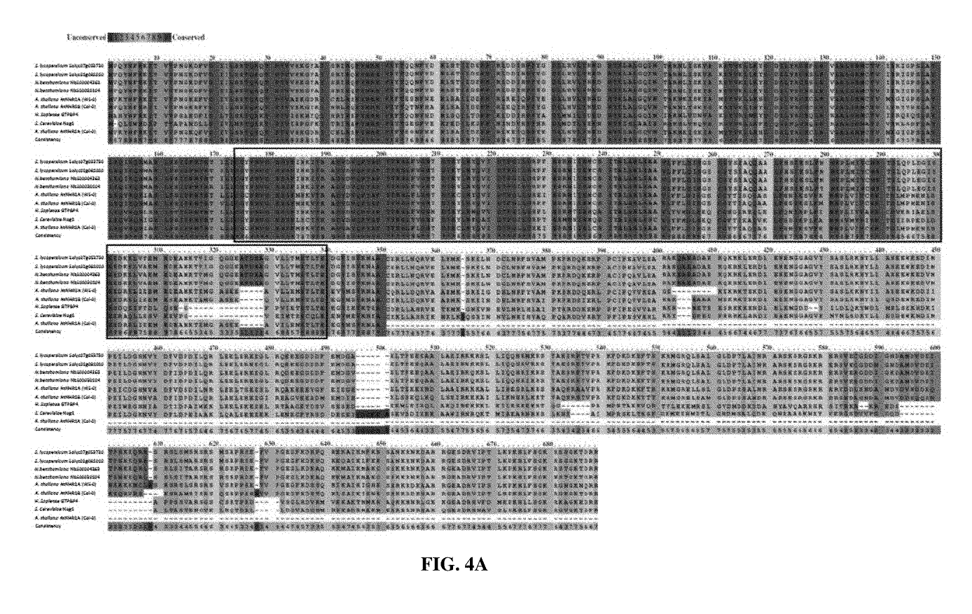

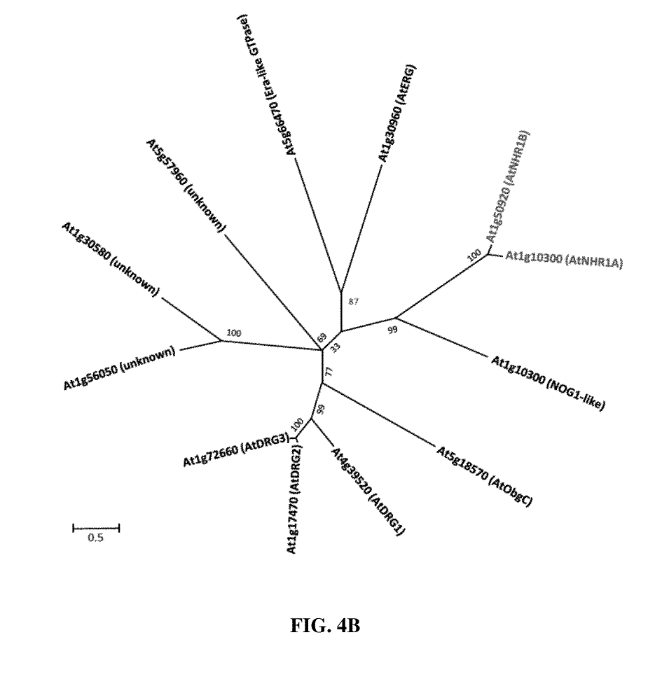

Two copies of full-length NHR1 with sequence similarities of 99.3% and 98.1% were identified in N. benthamiana and tomato, respectively (FIG. 4A). Two homologs of NbNHR1 were also identified in Arabidopsis, At1g10300 (named AtNHR1A; nucleotide sequence SEQ ID NO:1, encoded amino acid sequence SEQ ID NO:7) and At1g50920 (named AtNHR1B; nucleotide sequence SEQ ID NO:2, encoded amino acid sequence SEQ ID NO:8). AtNHR1A and AtNHR1B were 79% similar at the nucleotide level and 76% similar at the amino acid level. NbNHR1 showed a high degree of similarity to yeast nucleolar G protein 1 (Nog1) (42.7%) and human GTP binding protein 4 (GTPBP4) (48.6%), proteins belonging to the small GTP-binding family protein OBG (FIG. 4A).

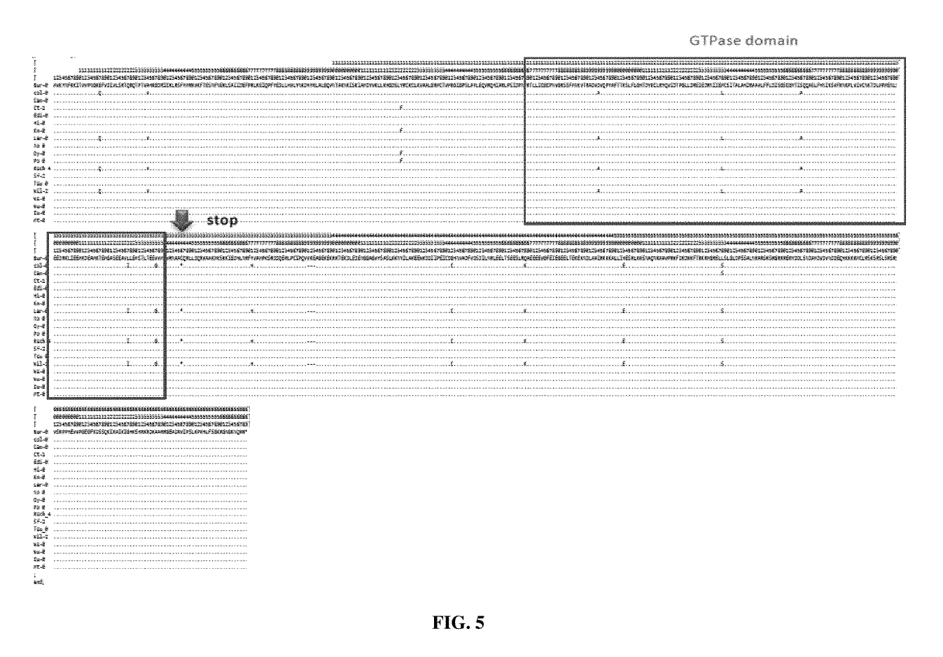

Annotation of the AtNHR1A sequence from The Arabidopsis Information Resource (TAIR) showed 2,064 bps containing two exons and one intron, and predicted to encode a protein of 687 amino acids. However, results from reverse transcription-PCR (RT-PCR) followed by sequencing showed that no intron is present in AtNHR1A and it encodes a truncated protein with 346 amino acids. The reason why TAR annotation shows the presence of an intron in AtNHR1A is due to the presence of a stop codon at the predicted intron. To investigate if the early termination occurs only in Col-0 or other Arabidopsis ecotypes, AtNHR1A amino acid sequences were examined in 19 different ecotypes. Interestingly, the truncated version of AtNHR1A is only present in four Arabidopsis ecotypes—Col-0, Ler-0, Rsch-4 and Wil-2 (Table 1). In contrast to NHR1A, NHR1B sequences were highly similar among different ecotypes. This early translational termination did not affect the GTPase domain in any of the Arabidopsis ecotypes (FIG. 5). Furthermore, sequence alignment with AtNHR1A homologs of other eukaryotes and the EST database of Arabidopsis suggested that the AtNHR1A start codon begins 87 bps downstream of the start codon annotated by TAIR. According to the protein expression result, the 87-bp deletion does not affect the full translation of AtNHR1A. This modified form of AtNHR1A was used for all experiments herein.

Full-length recombinant AtNHR1A was expressed in Rosetta E. coli (Novagen). Full-length Arabidopsis NHR1A cDNA was cloned into the pET59 vector (Novagen) to produce an N-terminal His-tagged fusion protein. Bacterial cells were grown in LB medium with 50 μg/mL carbenicillin to a density of OD₆₀₀=0.4-0.6. Expression of recombinant proteins was induced overnight at 19° C. with 0.2 mM IPTG. Proteins were extracted using CelLytic B cell lysis buffer (Sigma-Aldrich) and purified using Ni-NTA agarose (Qiagen). The expression of AtNHR1A protein was confirmed by Western blot using 6×His antibody.

The predicted domain for GTPase activity of AtNHR1 is highly conserved among different organisms. Using the GTPase domain sequence of AtNHR1A and AtNHR1B, a total of 10 Arabidopsis homologs were identified (FIG. 4B). Phylogenetic analysis revealed that AtNHR1A and AtNHR1B are highly similar to the small GTP-binding family proteins Obg, DRG and ERG of Arabidopsis (FIG. 4B).

TABLE 1
Arabidopsis ecotypes.

| | | AIMS Stock | Nucleotide Sequence |
|---|---|---|---|
| Accession | Origin | Centre No. | (nt 1100 to 1102) |
| Bur-0 | Ireland | CS6643 | TGT |
| Can-0 | Canary Isles | CS6660 | TGT |
| Ct-1 | Italy | CS6674 | TGT |
| Edi-0 | Scotland | CS6688 | TGT |
| Hi-0 | Netherlands | CS6736 | TGT |
| Kn-0 | Lithuania | CS6762 | TGT |
| Ler-0 | Poland, formerly | CS20 | TGA |
| | Germany | | |
| Mt-0 | Libya | CS1380 | TGT |
| No-0 | Germany | CS6805 | TGT |
| Oy-0 | Norway | CS6824 | TGT |
| Po-0 | Germany | CS6839 | TGT |
| Rsch-4 | Russia | CS6850 | TGA |
| Sf-2 | Spain | CS6857 | TGT |
| Tsu-0 | Japan | CS6874 | TGT |
| Wil-2 | Russia | CS6889 | TGA |
| Ws-0 | Russia | CS6891 | TGT |
| Wu-0 | Germany | CS6897 | TGT |
| Zu-0 | Germany | CS6902 | TGT |
| Col-0 | Columbia | CS1092 | TGA |

### Example 9

### AtNHR1A and AtNHR1B are Induced in Response to Biotic and Abiotic Stresses.

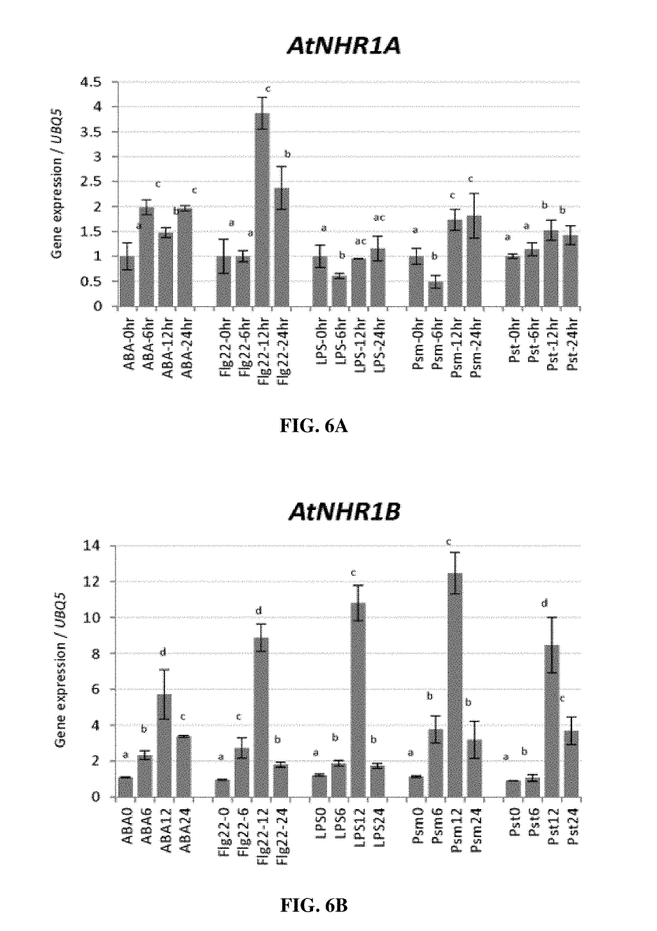

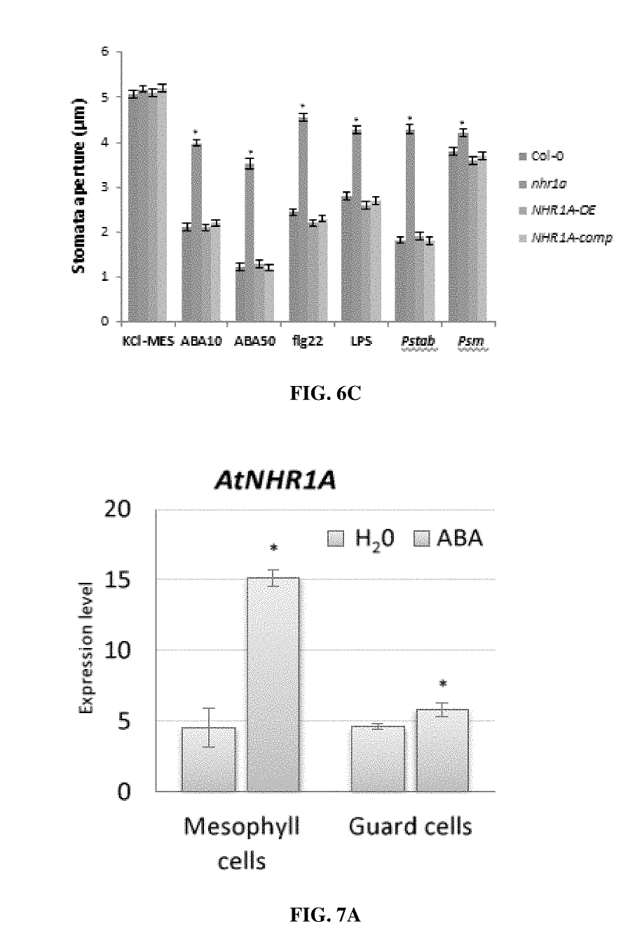

The gene expression patterns of AtNHR1A and AtNHR1B were determined by quantitative RT-PCR (qRT-PCR) after treating wild-type Col-0 plants with ABA, PAMPs (Flg22 and LPS), host (P. syringae pv. maculicola) and nonhost (P. syringae pv. tabaci) bacterial pathogens. Arabidopsis wild-type (Col-0) plants were individually syringe-infiltrated with ABA (10 μM), Flg22 (20 μM), or LPS (100 ng), or flood-inoculated with the pathogens P. syringae pv. maculicola (Psm) and P. syringae pv. tabaci (Pst) at 1×10⁴ cfu/mL. RNA was isolated from tissue samples harvested at 0 hrs, 6 hrs, 12 hrs and 24 hrs, and qRT-PCR was performed. AtNHR1A was induced ˜fourfold at 12 hrs post treatment (hpt) with Flg22, twofold with ABA treatment at 6 hpt and ˜1.5-fold after treatment with either the host or nonhost pathogens tested (FIG. 6A). AtNHR1B expression was highly induced at 12 hpt with ABA, Flg22, host and/or nonhost pathogens (FIG. 6B). Interestingly, at 24 hpt, the induction of AtNHR1B was reduced dramatically—by more than 50% (FIG. 6B).

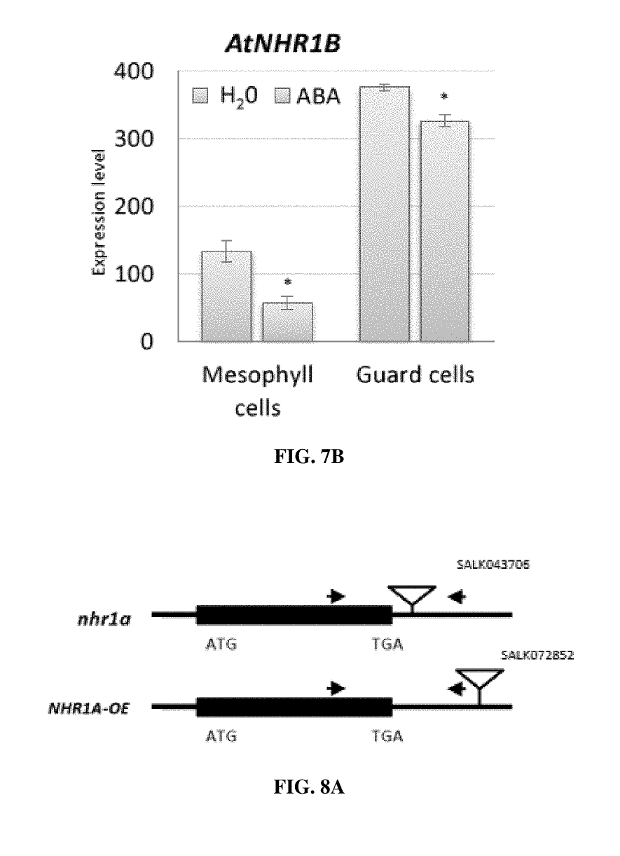

Since ABA is tightly associated with stomatal function, we used the publicly available Arabidopsis database to investigate the expression of AtNHR1A and AtNHR1B in stomatal guard cells (FIG. 7A and FIG. 7B). The transcript level of AtNHR1A in wild-type Col-0 was approximately threefold higher in mesophyll cells after ABA treatment compared to a water-treated control, but only a slight increase in AtNHR1A transcripts was observed in guard cells after ABA treatment (FIG. 7A and FIG. 7B). AtNHR1B transcript levels were significantly higher in both mesophyll cells and guard cells after water or ABA treatment compared to AtNHR1A. Interestingly, the pattern of AtNHR1B expression was different than AtNHR1A as the transcripts of AtNHR1B decreased after ABA treatment compared to water treatment in both mesophyll cells and guard cells (FIG. 7A and FIG. 7B).

Furthermore, transgenic Arabidopsis lines expressing the β-glucuronidase (GUS) reporter gene (Jefferson et al., 1987) under the control of AtNHR1A or AtNHR1B promoters were developed to determine AtNHR1A and AtNHR1B expression patterns in different plant tissues. β-glucuronidase (GUS) expression driven by AtNHR1A and AtNHR1B promoters were examined in one-week old and two-week old seedlings expressing either AtNHR1A or AtNHR1B promoter fusions to GUS, grown on 1×MS medium. GUS expression was seen in guard cells, hydathodes, floral parts, nectarines at the base of an early developing silique, and throughout a maturing silique and anther. Strikingly, AtNHR1A and AtNHR1B exhibit distinct patterns of expression in different tissues although both genes are strongly expressed in stomata. pAtNHR1A::GUS and pAtNHR1B::GUS expressions were also determined in 2-week-old seedlings after treatment with either ABA or PAMPs, or the host or nonhost pathogens. Consistent with the qRT-PCR results, pAtNHR1A::GUS and pAtNHR1B:: GUS expressions were detectable in all treatments. pAtNHR1B::GUS expression was more strongly induced after inoculation with the nonhost pathogen P. syringae pv. tabaci than with the host pathogen. These results indicate that AtNHR1A and AtNHR1B expression is modulated during plant defense responses.

### Example 10

### AtNHR1A is Necessary for the Regulation of Stomatal Closure in Response to Pathogens and Abiotic Stimuli.

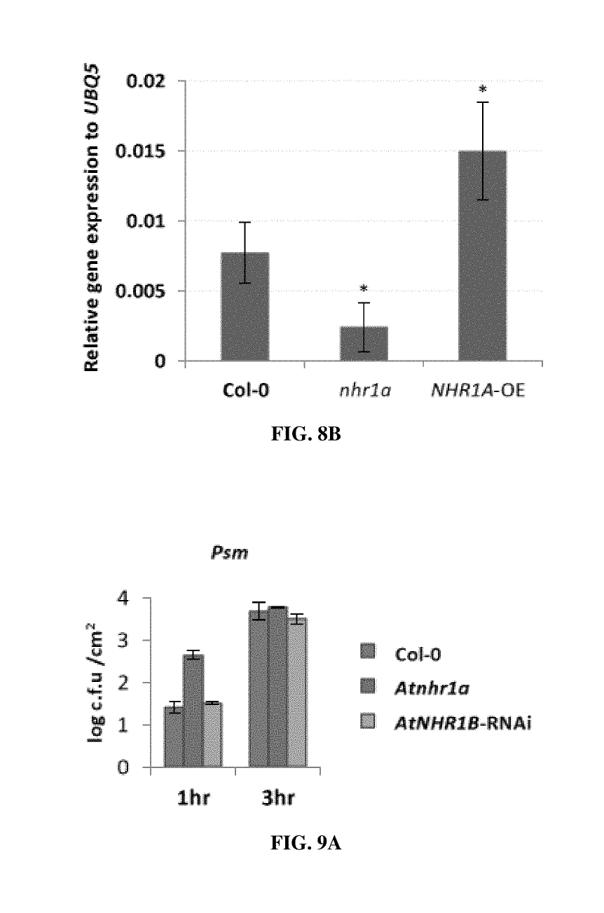

Two different T-DNA insertion mutants for AtNHR1A, SALK_043706 and SALK_072852, were identified and obtained from the Arabidopsis Biological Resource Center (FIG. 8A). Transcript analysis using RT-PCR demonstrated that AtNHR1A expression is absent in SALK_043706. Surprisingly, the transcripts of AtNHR1A in the SALK_072852 mutant were much higher than in the wild-type Col-0 (FIG. 8B). Further analysis of this mutant revealed that the T-DNA insertion is in a micro-RNA binding site in 3′ UTR, thus causing overexpression of AtNHR1A. Hereafter, this mutant will be considered as an AtNHR1A overexpresssor line (AtNHR1A-OE). Atnhr1a was transformed with a construct containing the AtNHR1A native promoter and coding region but without 3′ UTR for a complementation experiment. AtNHR1A expression in the complementing line (AtNHR1A-comp) was equivalent to the expression level of the AtNHR1A-OE. Western blot analysis showed a significant reduction of NHR1A in an nhr1a mutant as compared to Col-0. Membranes were incubated with anti-GTPBP4 (human) antibodies. Protein expression was examined in two different plant samples. Rubisco stained with Coomassie Brilliant Blue was used as a loading control.

To monitor stomatal function, Arabidopsis epidermal peels were prepared from wild-type Col-0, Atnhr1a, AtNHR1A-OE and AtNHR1A-comp plants, and were treated with stomata-opening buffer (KCl-MES), ABA (50 μM), flg22 (20 μM), LPS, nonhost pathogen P. syringae pv. tabaci and host pathogen P. syringae pv. maculicola at 1×10⁴ cfu/mL. In response to ABA, Flg22 and the nonhost pathogen P. syringae pv. tabaci, NHR1A-OE and AtNHR1A-comp lines closed stomata similarly to Col-0, while the Atnhr1a stomata remained open irrespective of the treatments. Treatment with the host pathogen P. syringae pv. maculicola caused stomata to remain open in all the lines tested. Quantification of these results was obtained by measuring the stomatal aperture (FIG. 6C). The aperture size of stomata in Col-0, AtNHR1AOE and AtNHR1A-comp lines decreased by 50 to 80% upon treatments that close stomata, while stomatal aperture in the Atnhr1a mutant was only reduced by 10% to 30% (FIG. 6C).

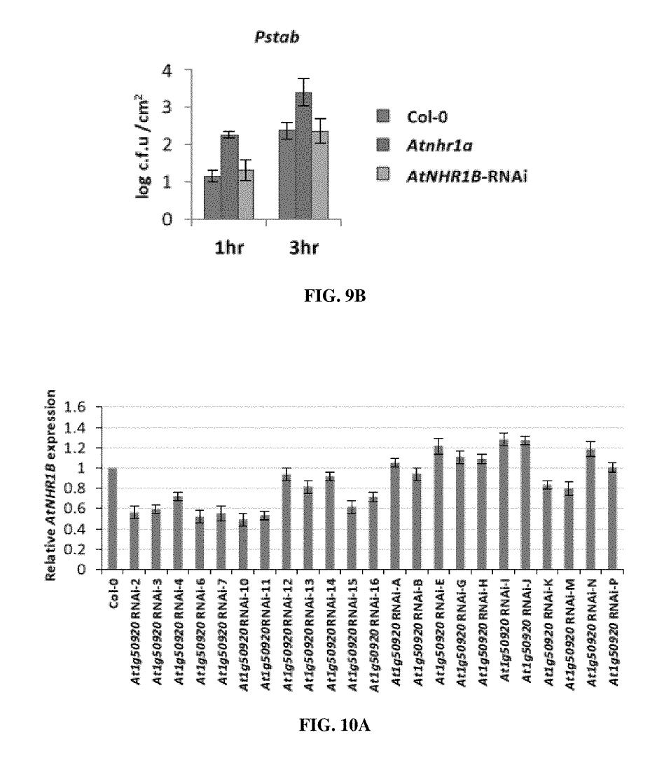

The fact that the Atnhr1a mutant was defective in closing stomata triggered by PAMPs and nonhost pathogens indicated that Atnhr1a could enable more pathogen entry. To test this, epidermal peels of Atnhr1a and Col-0 were individually incubated with the host and nonhost pathogens, P. syringae pv. maculicola (Psm; FIG. 9A) and P. syringae pv. tabaci (Pstab; FIG. 9B) expressing GFPuv (Wang et al., 2007), respectively. Bacterial entry was quantified in Atnhr1a and Col-0 plants at 1 hour(s) post-infection (hpi) and 3 hpi. Detached Arabidopsis leaves were floated in bacterial suspensions. After infection, leaves were surface-sterilized with 10% bleach, ground, serially diluted, and plated. The number of nonhost bacterial cells inside Atnhr1a mutant leaves was 10-fold higher than in Col-0 (FIG. 9B). The number of host bacterial cells was more in the Atnhr1a mutant at 1 hpi but was not different than wild-type at 3 hpi since the host pathogen was able to reopen stomata in both Atnhr1a and Col-0 (FIG. 9A). Similar results were also found in NHR1-silenced N. benthamiana plants (FIG. 9A and FIG. 9B).

### Example 11

### AtNHR1B is not Involved in Stomatal Defense, but it is Required for Nonhost Resistance Against Bacterial Pathogens.

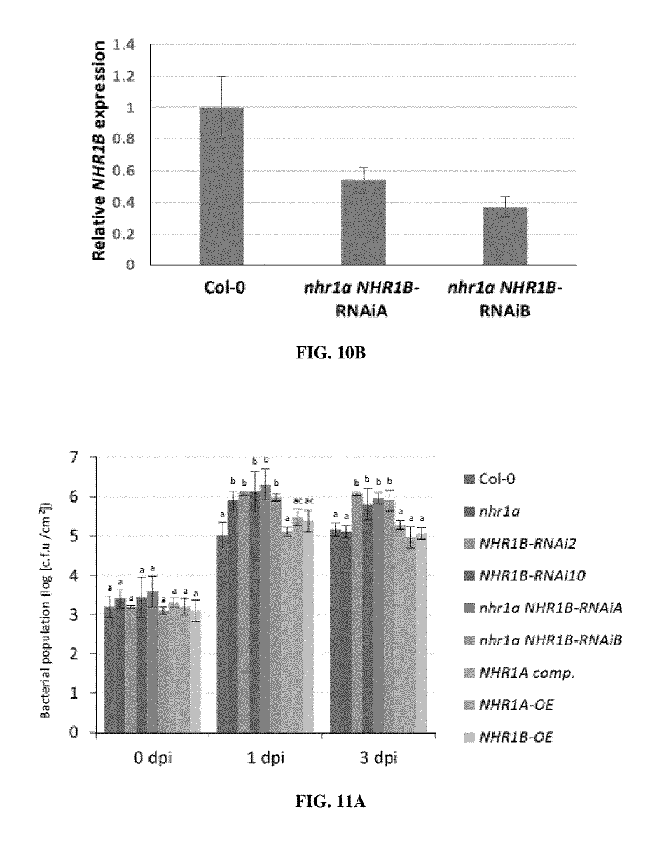

As shown above, NbNHR1- and SlNHR1-silenced N. benthamiana and tomato plants, respectively, compromise nonhost resistance. As Atnhr1b T-DNA insertion mutants were not available, to investigate if AtNHR1A and AtNHR1B also play a role in nonhost resistance, RNA interference (RNAi) lines were generated to downregulate AtNHR1B expression. 23 T₁ plants containing an AtNHR1B RNAi transgene were tested for AtNHR1B expression by qRT-PCR. Two RNAi lines, RNAi2 and RNAi10, that showed the greatest (˜50%) downregulation of AtNHR1B (FIG. 10A) were selected for further experiments. Similar to NbNHR1- and SlNHR1-silenced plants that showed stunted growth, AtNHR1B-RNAi plants were slightly smaller than wild-type (Col-0). However, the Atnhr1a mutant did not show a stunted phenotype. A double-mutant mimic was generated by transforming the Atnhr1a mutant with an AtNHR1B-RNAi construct. Two double-mutant mimics, nhr1a NHR1B-RNAiA and nhr1a NHR1B-RNAiB, that showed the highest level of AtNHR1B downregulation were selected for further experiments (FIG. 10B).

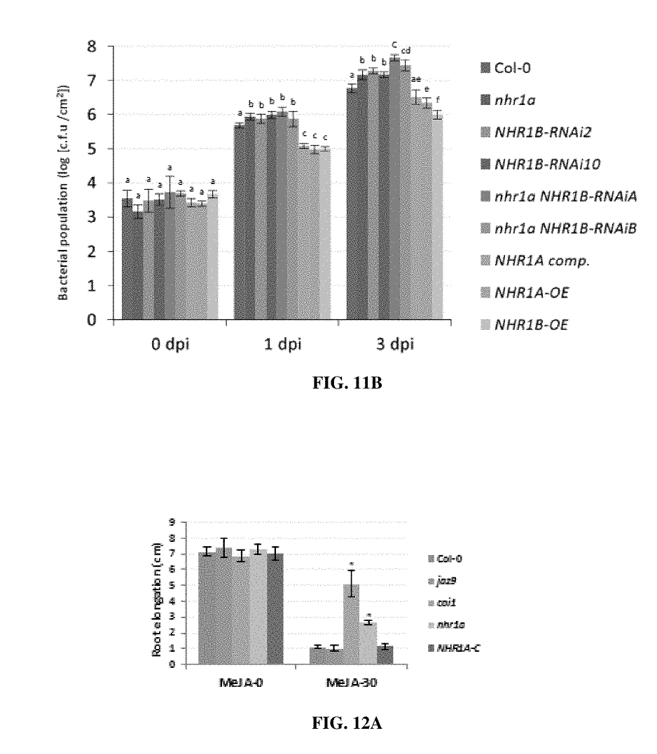

The double-mutant mimic, along with Col-0, single mutants and overexpressor lines, were flood-inoculated (Ishiga et al., 2011) with the nonhost pathogen P. syringae pv. tabaci (FIG. 11A) and the host pathogen P. syringae pv. maculicola (FIG. 11B). AtNHR1B-RNAi lines and the double-mutant mimic showed enhanced susceptibility to P. syringae pv. tabaci and had ˜10-fold increased bacterial growth when compared to Col-0 (FIG. 11A). By contrast, the Atnhr1a mutant did not compromise nonhost resistance even though ˜10-fold increase in bacterial growth was observed only at 1 dpi (due to more entry of bacteria) when compared to Col-0 (FIG. 11A). Both Atnhr1a and AtNHR1B-RNAi lines showed slightly enhanced susceptibility to the host pathogen P. syringae pv. maculicola by supporting higher bacterial growth (FIG. 11B). Double-mutant mimic lines showed an additive effect in comparison with single mutants for hyper-susceptibility to host pathogen inoculation. Strikingly, AtNHR1A comp, AtNHR1AOE and AtNHR1B overexpression lines (AtNHR1BOE) exhibited fewer disease symptoms and harbored less bacteria compared to Col-0 (FIG. 11B).

### Example 12

NHR1A Interacts with JAZ9 that is Involved in Stomatal Closure Through JA Signaling Pathway.

To determine the signaling components of AtNHR1A-mediated stomatal closure, an Arabidopsis yeast two-hybrid library was screened to identify proteins that interact with AtNHR1A. A total of 29 interacting proteins with AtNHR1A were identified, among those was the Jasmonate-Zim-Domain Protein 9 (JAZ9, At1g70700). Given the crucial function of JAZ proteins in the guard cell signaling pathway of Arabidopsis (Jammes et al., 2009; Niu et al., 2011), the relationship between JAZ9 and AtNHR1A was investigated. Colonies from a yeast two-hybrid (Y2H) prey vector expressing full-length AtNHR1A (1-360) co-transformed with a Y2H bait vector expressing JAZ9 or JAZ9Δjas and plated on synthetic complete (SC) media lacking leucine, tryptophan, and histidine, and containing X-Gal (5-bromo-4-chloro-3-indolyl-beta-D-galacto-pyranoside) were examined to detect interaction by the development of blue-colored colonies. Full-length AtNHR1A (1-360 aa) was found to interact with full-length JAZ9.

A bimolecular fluorescence complementation (BiFC) assay (Martin et al., 2009) was used to investigate the interaction of AtNHR1A with JAZ9 in vivo. Transient co-expression of AtNHR1A fused to the N- or C-terminal half of the enhanced yellow fluorescent protein (EYFP) with JAZ9 fused to the N- or C-terminal half of EYFP in N. benthamiana reconstituted YFP fluorescence, indicating in planta interaction of these proteins.

The C-terminal region of JAZ proteins containing the JA-associated (Jas) motif is required for interactions with COI1, MYC2 and other major proteins required for hormonal defense signaling (Wager and Browse, 2012). JAZ9 co-immunoprecipitates with NHR1A from plant extracts. To examine the interaction between NHR1A and JAZ9, His-tagged NHR1A protein expressed in E. coli was purified and mixed with total protein extracts from Col-0 or HA-JAZ9 expressing transgenic plants and was later incubated with anti-HA agarose conjugating resin. Anti-GTPBP4 antibody was used to detect NHR1A protein. Consistent with this report, JAZ9 without the Jas motif (JAZ9Δjas) did not interact with AtNHR1A. Because the Jas motif is conserved in the 12 JAZ proteins present in Arabidopsis, all the JAZ proteins were tested for interaction with AtNHR1A, as well as the sub-cellular localization of AtNHR1A in Arabidopsis. Yeast clones were grown in quadrate dropout media (-Leu, -Try, -His, -Ura) containing X-gal. NHR1A was cloned into bait plasmid pDEST32 and co-transformed with each JAZ protein (prey, pDEST22). Interestingly, it was found that JAZ1, JAZ3, JAZ4, JAZ5, JAZ9 and JAZ12 proteins also interact with NHR1A. AtNHR1A-GFP localized to nuclei and guard cells in one-week old Arabidopsis seedlings. These results indicate that interaction with NRH1A is associated with Jas domain of JAZ proteins.

This finding also indicates a redundant function of JAZ proteins for stomatal signaling associated with NHR1A. This is consistent with the finding that a jaz9 mutant does not show an obvious JA-related phenotype (Demianski et al., 2012; Thines et al., 2007). In addition, it has been reported that MYC2 interacts with all 12 JAZ proteins, further suggesting their redundant function (Fernandez-Calvo et al., 2011) in Arabidopsis.

### Example 13

### NHR1A can be Involved in the Regulation of JAZ9 Binding to COI1 for Stomatal Closure.

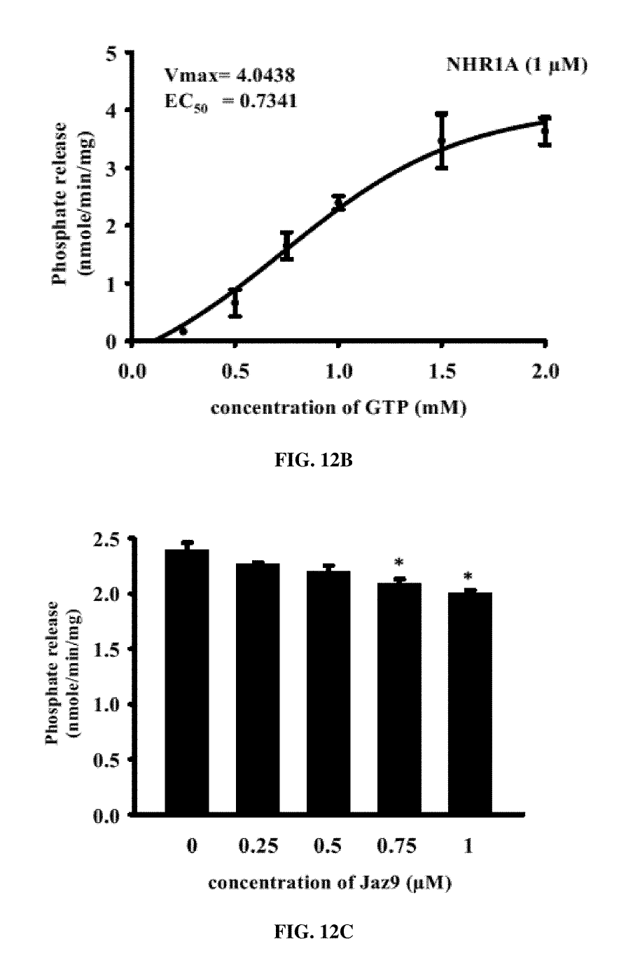

Several JAZ proteins, such as JAZ1, JAZ2, JAZ3, JAZ6, JAZ9 and JAZ10, have been known to directly interact with COI1 in Arabidopsis (Chini et al., 2009; Melotto et al., 2008; Thines et al., 2007; Yan et al., 2009; Zhou et al., 2013). As shown above, NHR1A directly interacts with JAZ1, JAZ3, JAZ4, JAZ5, JAZ9 and JAZ12. Without being limited by theory, the present inventors hypothesize that the function of JAZ9 can be modified by binding NHR1A, and this may affect COI1-mediated signaling for stomatal closure. The fast agro-mediated seedling transformation (FAST) assay was used in Col-0 and nhr1a to investigate whether NHR1A was required for JAZ9-COI1 interaction. Interestingly, the intensity of the interaction of JAZ9 with COB was greater in the nhr1a mutant than the intensity observed in Col-0. This result indicated that binding of NRH1A to JAZ9 modulates the interaction between JAZ9 and COB in Arabidopsis and can regulate JA-mediated defense signaling for stomatal opening and closure in response to bacterial pathogens. It was found that nhr1a is less sensitive to JA than Col-0, where roots were measured 7 days after seeds of different Arabidopsis lines were grown in MS medium plates with or without 30 μM of MeJA (FIG. 12A).

To further examine the role of AtNHR1A, GTPase activity of AtNHR1A was assessed using purified protein in a fluorescence-based assay (Willard et al., 2005). AtNHR1A has GTPase activity (FIG. 12B). Interestingly, in the presence of JAZ9, the rate of GTP hydrolysis significantly decreased (FIG. 12C). This finding was quantified by measuring the phosphate (Pi) release, using the ENZchek® phosphate assay kit (Invitrogen®), after incubating NHR1A with different concentrations of JAZ9. At concentrations of 0.75 μM and 1 μM of JAZ9, there was a reduction of 20% in phosphate release compared with the phosphate release of AtNHR1A without JAZ9 (FIG. 12C).

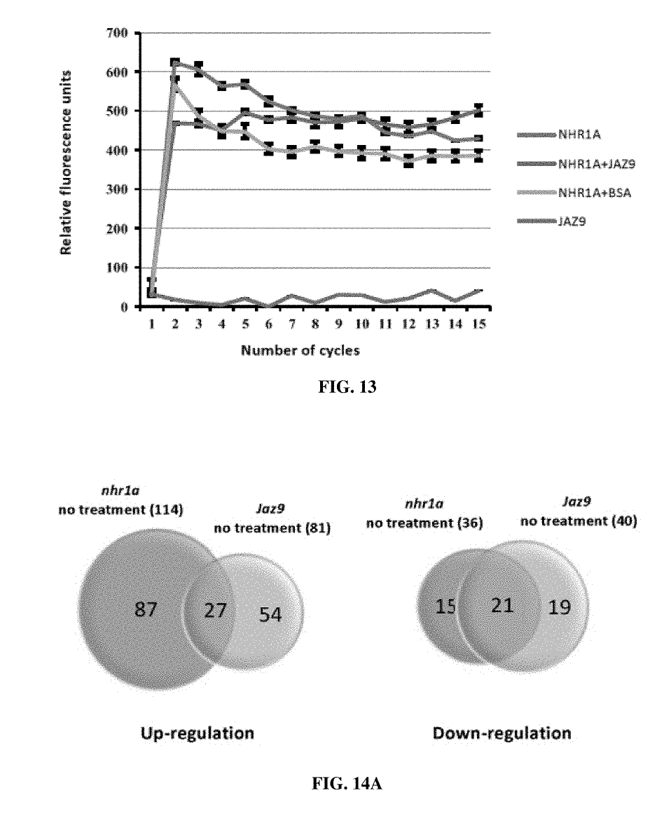

The GTPase activity of AtNHR1A was reduced when JAZ9 was present (FIG. 13), indicating that the binding of JAZ9 to AtNHR1A maintains GTPase activity of AtNHR1A that can be capable of recruiting or remodeling proteins, which is important for guard cell signaling. GTP binding and hydrolysis by AtNHR1A protein was measured using GTP-BODIPY-FL in real-time fluorescence assays in the presence of absence of JAZ9 protein. Phosphate production was detected as a change in absorbance at 360 nm and the amount of Pi released was estimated from the corresponding values obtained with a standard curve. Data were plotted as nanomoles of Pi released/min/mg and only for NHR1A; data were fitted using nonlinear regression in SigmaPlot 11.0. Without being limited by theory, the present inventors hypothesize that the GDP-bound form of AtNHR1A is not able to fulfill this function.

### Example 14

### AtNHR1A Positively Regulates JA- and ABA-Mediated Guard Cell Signaling in Arabidopsis.

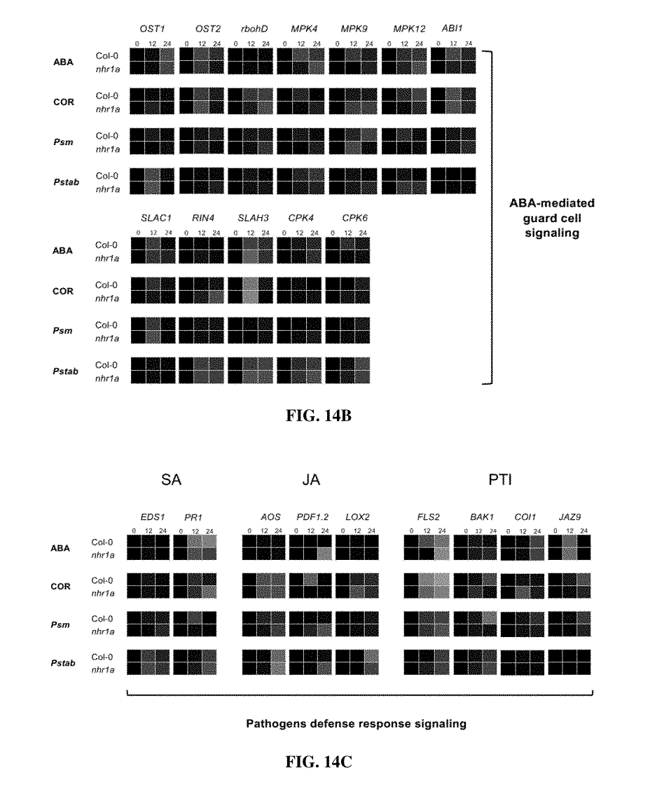

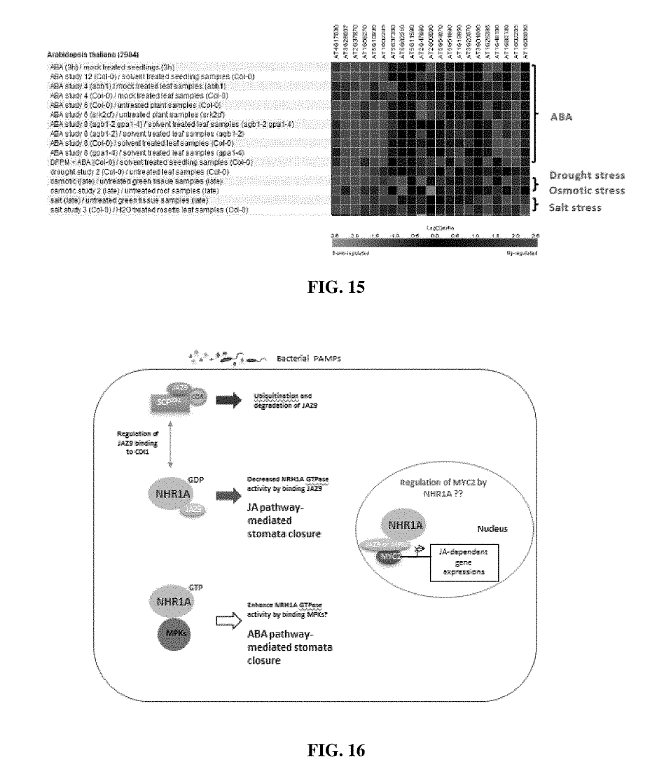

Microarray analysis was performed in Col-0, and nhr1a and jaz9 mutants to further determine the function of NHR1A in guard cell signaling. A total of 114 and 81 genes were up-regulated, and 36 and 40 genes were down-regulated, respectively, in nhr1a and jaz9 mutants compared to Col-0 (FIG. 14A). Interestingly, 21 down-regulated genes were common in both the nhr1a and jaz9 mutants, suggesting that NHR1A and JAZ9 may follow more or less the same signaling pathway. Most of the genes commonly down-regulated in nhr1a and jaz9 are highly responsive to ABA and drought stresses, indicating the functional relationship of NHR1A and JAZ9 for the stomatal signaling pathway (FIG. 15). Furthermore, the expression patterns of 21 genes commonly down-regulated in nhr1a and jaz9 were compared to the Arabidopsis microarray database to identify microarray data similar to nhr1a and jaz9. The nhr1a mutants were less sensitive to ABA and tolerant to drought stress. Col-0 and nhr1a plants were grown for four weeks (21° C. with a 14 hrs day, and 18° C. with a 10 hrs night), then plants were dehydrated until drought symptoms appeared. After leaves were completely collapsed, plants were re-watered to revival. Seedlings were grown for two weeks in MS without ABA (1 μM). Results indicate that NHR1A can be involved in the regulation of MYC2 and the JAZ-mediated JA signaling pathway.

To determine the relationship of NHR1A to other genes involved in guard cell signaling, qRT-PCR analysis was performed to determine expression levels of the guard cell signaling genes (OST1, OST2, rbohD, MPK4, MPK9, MPK12, ABI1, SLAC1, RIN4, SLAH3, CPK4 and CPK6) upon exposure to both abiotic and biotic stimuli. Three-week-old Arabidopsis seedlings grown in MS medium were inoculated with ABA, COR, P. syringae pv. maculicola and P. syringae pv. tabaci, and samples were collected 0 hrs, 12 hrs, and 24 hrs after inoculation for RNA extractions. qRT-PCR analysis was performed with three biological and technical replications. After ABA treatment, a majority of the genes tested were differentially expressed in nhr1a (FIG. 14B). Interestingly, upon COR treatment, expression for all genes tested, except ABI1, was altered in nhr1a compared to Col-0. In addition, OST1, OST2, MPK4, MPK9 and MPK12 were differentially expressed in nhr1a after host and nonhost pathogen inoculations compared to Col-0. Without any treatments, the expression levels of genes tested were not significantly different in nhr1a compared to Col-0.

The expression patterns of several marker genes for SA and JA pathways were also examined after different treatments. Two genes EDS1 (enhanced disease susceptibility 1) and PR1 (pathogenesis-related 1), representing the SA-mediated defense pathway, showed somewhat similar patterns of expression in Col-0, nhr1a and NHR1AOE lines after different treatments. However, three genes, AOS (allene oxide synthase), PDF1.2 (plant defensin 1.2) and LOX2 (lipoxygenase 2), representing the JA pathway, were differentially expressed in nhr1a compared to Col-0 in different treatments. Furthermore, the expression of genes involved in PAMP-triggered immunity (PTI), BAK1 and stomatal defense, COI1 and JAZ9, was altered in nhr1a compared to Col-0 after COR and pathogen treatments (FIG. 14C). This result indicates that the lack of NHR1A modifies JA- and PTI-mediated defense pathways. Collectively, these findings indicate NHR1A is the key regulator for stomatal closure that mediates cross-talk between JA and ABA hormonal signaling pathways.

FIG. 16 shows a model of NHR1A function in stomata-mediated defense response to abiotic and biotic stimuli. COI1 recruits JAZ9 for ubiquitination and degradation in the presence of COR/JA. NHR1A interacts with JAZ9 for regulating JA-mediated stomata closure in response to bacterial pathogens but acts in a pathway independent of ABA. NHR1A can also be involved in MAP kinases-mediated ABA signaling pathway for stomatal open/closure. NHR1A localizes to nuclei like JAZ9 and MYC2. NHR1A can participate in the cross-talk between JAZ9 and MYC2 for regulating JA signal transduction pathway.

### Example 15

Silencing of GCN4 in Nicotiana benthamiana Compromises Nonhost Resistance.

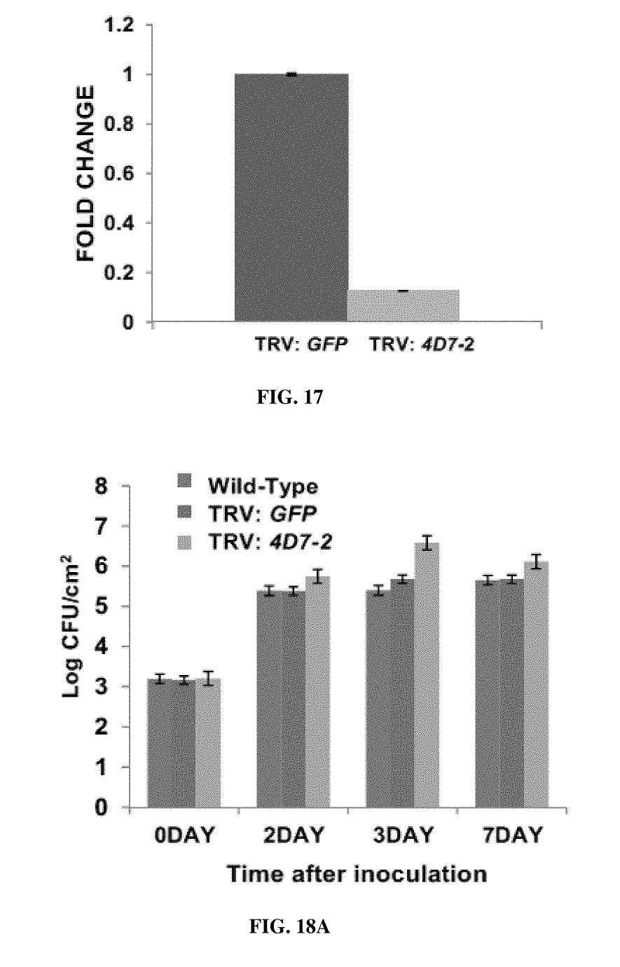

Another cDNA clone identified as a component of nonhost resistance using VIGS-mediated screening of a normalized cDNA library (Anand et al., 2007, Rojas et al., 2012, Wangdi et al., 2010) was named TRV:4D7-2. When the endogenous copy of this gene was silenced in N. benthamiana, plants showed stunted growth and thick curled brittle leaves phenotype. Silenced plants (TRV:4D7-2) had ˜85% down regulation of 4D7-2 mRNA as shown by qRT-PCR (FIG. 17). The 4D7-2-silenced plants when challenged by vacuum infiltration at 1×10⁴ cfu/mL with the nonhost pathogens Pseudomonas syringae pv. tomato T1, P. syringae pv. glycinea and Xanthomonas campestris pv vesicatoria showed disease symptoms characterized by leaf necrosis and chlorosis. Development of the HR was also apparent 1, 2, and 3 dpi when 4D7-2-silenced plants were challenged by syringe-infiltration with the nonhost pathogens P. syringae pv. tomato T1 and P. syringae pv. maculicola at 10⁸ cfu/mL.

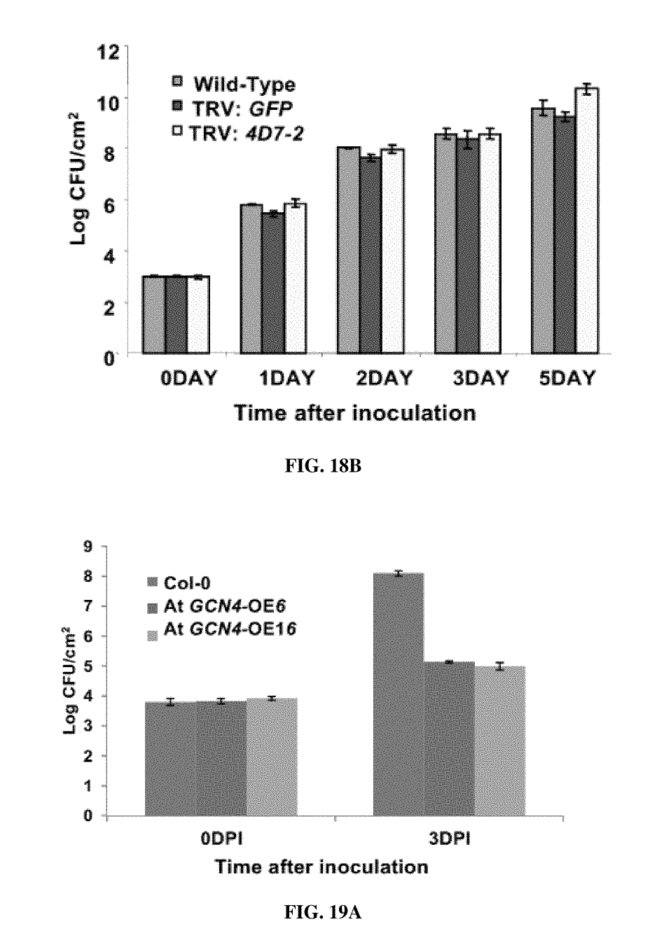

The 4D7-2 silenced plants showed more bacterial colonization after infiltration at a concentration of 1×10⁴ cfu/mL of GFPuv-labeled nonhost pathogen Pseudomonas syringae pv. tomato T1 (Wang et al., 2007) and showed up to 10-fold more bacterial growth 3 dpi as compared with wild-type plants and non-silenced controls (TRV:4D7-2) (FIG. 18A). After infiltration at a concentration of 1×10⁴ cfu/mL with the host pathogen P. syringae pv. tabaci, which normally grows and causes disease in wild-type plants, 4D7-2-silenced plants become hyper-susceptible to this pathogen and showed more colonization after infiltration with P. syringae pv. tabaci (GFPuv) (Wang et al., 2007) and supported higher bacterial growth (˜10-fold) 5 dpi in comparison with wild-type plants (FIG. 18B). Furthermore, after syringe-inoculation with the nonhost pathogens P. syringae pv. tomato T1 and P. syringae pv. maculicola at high-doses of inoculum to promote the development of the HR, 4D7-2-silenced plants showed a delayed HR. In non-silenced controls (TRV:GFP), the HR was observed 1 dpi, while the HR in 4D7-2-silenced plants appeared 3 dpi with P. syringae pv. tomato T1 and 2 dpi with P. syringae pv. maculicola.

### Example 16

Analyses of GCN4 Sequences in Nicotiana benthamiana and Arabidopsis.

Sequencing of the cDNA insert in TRV:4D7-2 clone revealed 93% nucleotide identity to putative ABC transporter F family member 4-like gene of tomato and 78% identity to Arabidopsis At3G54540 annotated as GCN4 (general control non-repressible 4), which is a member of the GCN sub family of ABC transporters proteins (Sanchez-Fernandez et al., 2001). The full-length GCN4 gene in N. benthamiana was cloned using the tomato GCN4 sequence to design PCR primers. The Arabidopsis thaliana GCN4 nucleotide sequence is provided as SEQ ID NO:9, and the encoded Arabidopsis thaliana GCN4 amino acid sequence is provided as SEQ ID NO:10.

The members of ABC transporter proteins of F-subfamily contain only nucleotide binding domains and not transmembrane domains and are therefore are not bona-fide transporters. Domain analysis using SMART revealed that this protein belongs to class 1 of AAA⁺ (ATPases associated with diverse cellular activities) proteins and contains two AAA⁺ modules (White & Lauring, 2007).

### Example 17

### GCN4 Overexpressing Arabidopsis are Pathogen Resistant.

Arabidopsis transgenic lines overexpressing GCN4 under a 2×-35S promoter were developed to investigate the role of GCN4. In order to determine whether GCN4 overexpression confers pathogen resistance, wild-type Col-0 and two GCN4 overexpressing lines were flood-inoculated (Ishiga et al., 2011) with the host pathogen P. syringae pv. maculicola at 2×10⁶ cfu/mL. Flood inoculation mimics the natural mode of infection in foliar pathogen as pathogens get entry into the apoplast through stomata. Five dpi, wild-type Col-0 developed disease symptoms, and 3 dpi, bacteria grew 1000-fold in Col-0. Strikingly, the GCN4 overexpressor lines did not have any disease symptoms and only grew ˜10-fold 3 dpi when compared to 0 dpi. Results showed a striking difference of 3 logs between wild-type Col-0 and the AtGCN4 overexpresssor lines (FIG. 19A). Surprisingly, however, syringe-infiltration did not show any significant difference between wild-type Col-0 and AtGCN4 overexpressor lines (FIG. 19B). These results indicate that the entry of pathogen through natural openings such as stomata is blocked in the AtGCN4 overexpresssor lines when compared to wild-type Col-0.

### Example 18

GCN4 Overexpressing Arabidopsis do not Reopen Stomata after Treatment with the Host Pathogen P. syringae pv. tomato DC3000 or Coronatine.

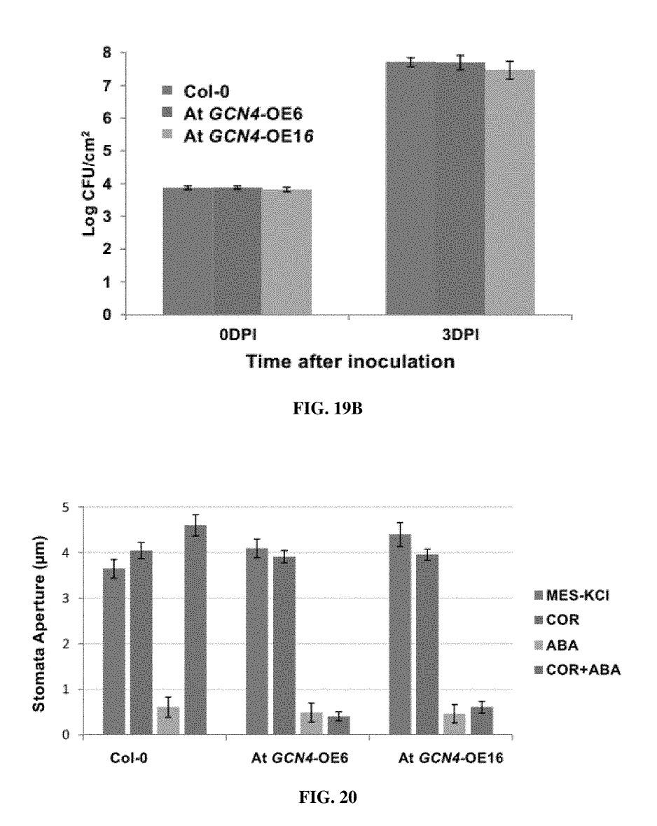

Upon detection of PAMPs, stomata rapidly closes to prevent entry of the pathogen into apoplast (Melotto et al., 2006; Lee et al., 2013). The host pathogen P. syringae pv tomato strain DC3000 produces a nonhost specific phytotoxin, coronatine (COR), which has been shown to reopen stomata 3 hpi (Zeng, 2010) in wild-type Col-0 plants. Epidermal peels of AtGCN4 overexpressing lines were used to investigate whether stomata reopen after treatment with MES buffer (stomata opening buffer; control) or the host pathogen P. syringae pv. tomato DC3000. In wild-type Col-0, stomata reopened 3 hpi, while in the AtGCN4 overexpressor lines (AtGCN4-OE6 and AtGCN4-OE16), stomata remained closed even 4 hpi with P. syringae pv. tomato DC3000. Stomatal aperture was measured after ABA and coronatine treatments in order to investigate stomatal function in AtGCN4 overexpressor lines. ABA treatment induces stomatal closure in plants. After ABA treatment, the stomatal aperture size was reduced due to closing in both wild-type Col-0 and the AtGCN4 overexpressor lines. Upon treatment with coronatine, re-opening of stomata was observed by increased aperture size in Col-0 plants. However, the stomatal aperture size did not increase in GCN4 overexpressor lines (FIG. 20). These results indicate that stomata of AtGCN4 overexpressor lines are insensitive to re-opening by P. syringae pv tomato DC3000 and purified coronatine.

### Example 19

AtGCN4 is Localized to Stomata and Interacts with SLAC1 and RIN4.

Stable transgenic lines were developed expressing GFP fused to the C-terminal end of AtGCN4 and driven under its native promoter. AtGCN4-GFP localized in guard cells, plasma membrane, and cytoplasm. Without being limited by theory, as AtGCN4 localized in the plasma membrane and guard cells, and plays role in the stomatal function, the present inventors reasoned that AtGCN4 interacts with other proteins responsible for stomatal function, such as SLAC1 and RIN4. SLAC1 closes stomata on ABA signaling (Vahisalu et al., 2008) and RIN4 activates a plasma membrane H⁺-ATPase and opens stomata (Liu et al., 2009). A yeast two-hybrid system between GCN4:SLAC1 and GCN4:RIN4 was used to investigate potential protein-protein interactions. AtGCN4 interacted with SLAC1 and RIN4. A BiFC assay (Hu et al., 2002) was used to verify these interactions in planta. AtGCN4 was co-transformed in yeast with either SLAC1 or RIN4 to observe protein-protein interaction using a yeast two-hybrid system. Interaction was observed in yeast growth on SD agar media (-His/-Leu/-Trp). The C-terminal half of yellow fluorescent protein (YFP) fused to the N-terminus of AtGCN4 (c-YFP-AtGCN4) and the N-terminal half of YFP fused to the C-terminus of SLAC1 (SLAC1-nYFP) were co-infiltrated into N. benthamiana for transient co-expression and observed 3 dpi. Protein-protein interactions were observed as yellow fluorescence with an equivalent bright field image. The C-terminal half of YFP fused to the N-terminus of AtGCN4 (c-YFP-AtGCN4) and the N-terminal half of YFP fused to the C-terminus of RIN4 (RIN4-nYFP) were co-expressed in N. benthamiana along with plasma membrane marker PM-rk. Protein-protein interactions were observed as yellow fluorescence while the plasma membrane was visualized as red fluorescence. SLAC1 and AtGCN4 interacted in the guard cells plasma membrane and cytoplasm while RIN4 interacted with AtGCN4 in the plasma membrane.

### Example 20

### Cell Type-Specific Expression Patterns of Arabidopsis GCN4.

The predicted promoter region of the GCN4 gene was fused to the coding region of the 3-glucuronidase (GUS) reporter gene, and transferred to a binary vector for stable transformation into Arabidopsis, to determine the cell type-specific expression patterns of AtGCN4. Using GUS staining, AtGCN4: GUS expression was analyzed in one-week-old seedlings and observed in cotyledons, leaves, and roots. AtGCN4: GUS expression was also analyzed 4-week-old mature plants and GUS expression was observed in the mature leaf and guard cells. pATGCN4:GUS was also expressed in the floret petals, sepals, stamens, and stigma tips. GUS staining was also seen in silique sheaths.

### Example 21

### GCN4 Overexpressor Lines are Drought Tolerant.

Stomata play an important role during drought conditions, therefore, the role of AtGCN4 in drought tolerance was investigated using a drought tolerance assay withholding water (Jiang et al., 2012). To simulate drought conditions, water was withdrawn for 9 days and rewatered normally thereafter. After 9 days of water withdrawal, wild-type Col-0 had severe drought phenotypes characterized by dried and wilted leaves, while the AtGCN4 overexpressor lines survived with dull but still green leaves. After re-watering, wild-type Col-0 did not survive while AtGCN4 overexpressor lines regained color and began recovering.

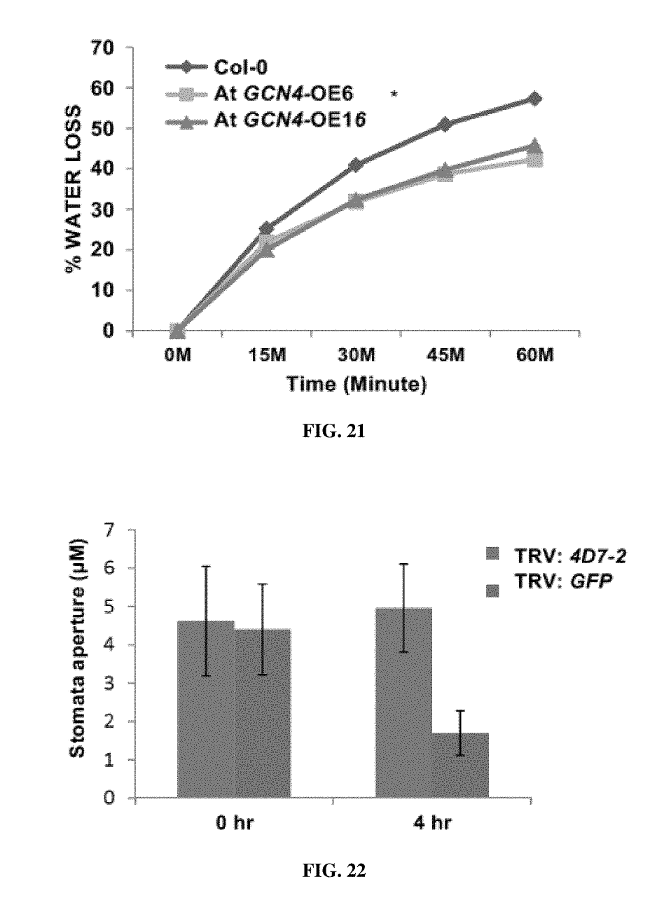

Transpirational water loss is an important factor associated with drought tolerance. Rossettes were detached and the change in the fresh weight was measured at 15 minutes intervals over 60 minutes (Jiang et al., 2012) in order to assess the rate of water loss in AtGCN4 overexpressor lines relative to wild-type Col-0 plants. The AtGCN4 overexpressing Arabidopsis plants showed a slower rate of water loss compared to wild-type Col-0 plants (FIG. 21).

### Example 22

GCN4 Silenced Plants in Nicotiana benthamiana have Defective Stomata.

The morphology of the stomata in NbGCN4-silenced lines was observed to investigate whether down regulation of NbGCN4 accounted for the compromise in nonhost disease resistance. In NbGCN4-silenced N. benthamiana plants, ˜40% of stomata in observed field areas had normal morphology while ˜60% had abnormal morphology consisting of either altered chloroplast organization or no chloroplasts. Generally, within one hour of bacterial infection, plants close their stomata as a defense response. Therefore, stomatal aperture was measured in the NbGCN4-silenced line (TRV:4D7-2) and compared with non-silenced controls (TRV:GFP) after inoculation with the non-host pathogen P. syringae pv tomato T1. Stomata in non-silenced controls (TRV:GFP) closed and the stomatal aperture was reduced by ˜75% 4 hpi (FIG. 22). By contrast, in silenced-plants (TRV:4D7-2), stomata remained open 4 hpi and the aperture size did not change relative to 0 hpi (FIG. 22).

### Example 23

### NHR1A, NHR1B and GCN4 Overexpressor Rice Lines are Drought Tolerant.

Drought is a major adverse environmental factor in most parts of the world causing substantial crop yield losses. Drought is predicted to become more severe and more widely distributed due to climate change. Rice is one of the staple foods for more than one-half of the world's population. It is quite sensitive to even mild drought stress and needs almost twice the amount of water compared to wheat or maize. Therefore, improvement of water use efficiency or drought tolerance is an important trait for enhanced rice production. Transgenic rice lines were created that constitutively over-express genes that are known to have an important role in stomatal aperture regulation and biotic stress tolerance. Transgenic lines showed an enhanced drought tolerance, an increased cell sap osmolality and abscisic acid level but decreased leaf water loss.

### A. AtNHR1A and AtNHR1B Over-Expression Leads to Drought Tolerance in Rice Plants.

In order to test the drought tolerance in rice, AtNHR1A and AtNHR1B over-expression and empty vector transformed control rice plants were grown in plastic nursery pots for 45 days under greenhouse condition. Real-time RT-qPCR expression analysis was performed to verify the overexpression of transgene and it revealed over thousand fold induction of AtNHR1A and AtNHR1B transcripts in AtNHR1A and AtNHR1B overexpressor lines respectively compared to the empty vector transformed control. Drought was imposed by withholding water. Soil moisture was continuously monitored and it dropped to 0% after 6 days of withholding water. Plants were kept for 11 days by withholding the water supply. Control plants showed dried, brittle and rolled leaves whereas there were still many green and half rolled leaves in AtNHR1A and AtNHR1B over-expression lines. Plants were rewatered on 11^{th} day after drought imposition. AtNHR1A and AtNHR1B over-expression lines recovered, plants turned green and started to grow whereas the control plants dried with a few green leaves.

### B. Evaluation of Physiological Parameters Revealed Enhanced Drought Tolerance of AtNHR1 and AtNHR1B Overexpressors.

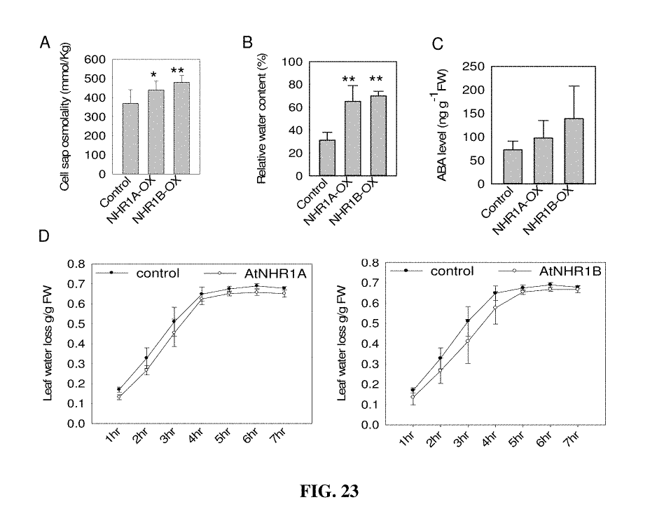

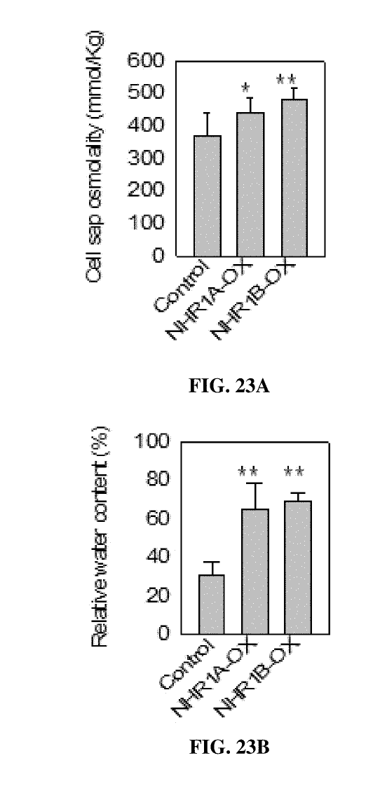

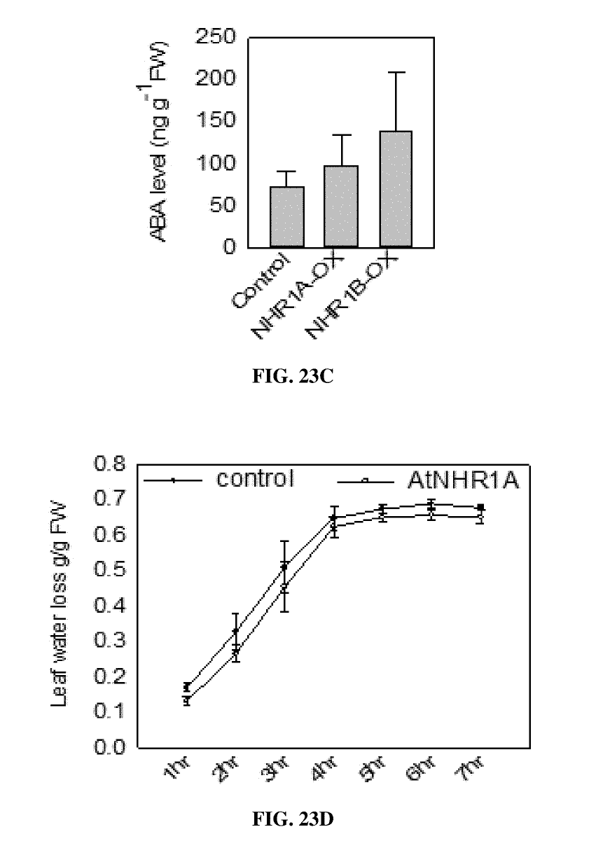

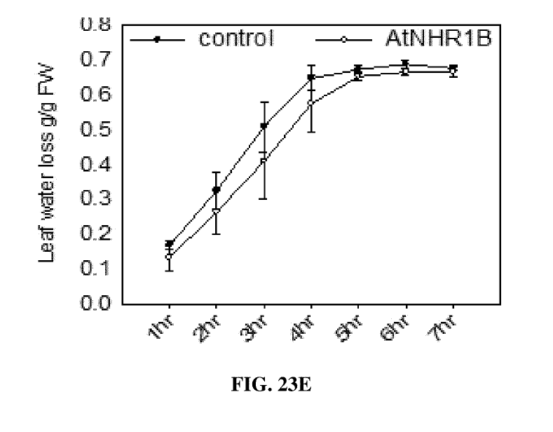

Physiological parameters were measured in NHR1B- and NHR1A-OX lines and it showed: (i) An increased cell sap osmolality (FIG. 23A) which can be due to increased organic solutes. Metabolite profiling in OX and control lines will unravel the altered organic solutes. An increased cell sap osmolality helps plant to lose less water but improve water uptake from soil. (ii) Higher leaf relative water content (RWC) (FIG. 23B). (iii) An increased ABA level (FIG. 23C) which could help plants to reduce water loss by closing stomata and inducing a significant increase in antioxidant enzymes and improving protein transport, carbon metabolism and expression of resistance proteins. (iv) Lower leaf water loss (FIG. 23D) that helps plants to conserve water.

### C. OsGCN4 Over-Expression Leads to Drought Tolerance in Rice Plants.

The rice ortholog of the Arabidopsis GCN4 DNA sequence (OsGCN4; SEQ ID NO:11) was over-expressed in rice variety Kitaake. In order to evaluate the drought tolerance, Os-GCN4 over-expressers and wild-type plants were grown in plastic pots under controlled condition in a growth chamber. Drought was imposed on 24-d-old plants by withholding water supply. Soil moisture was continuously monitored which decreased continuously and dropped to zero after seven days of drought imposition. After 11 days of drought imposition, leaves of the control wild-type plants were dry and rolled whereas the most of the leaves of Os-GCN4 over-expressers were still green although these were rolled. Plants were rewatered on 11^{th} day and after four days of rewatering, OsGCN4 over-expresser plants recovered with most of the leaves turning green and opened whereas the wild-type control leaves were dry with a few partly green leaves.

What is claimed is:

1. A method of increasing drought tolerance and resistance to bacterial infection comprising overexpressing a NHR1 or GCN4 gene, or both, in a plant, wherein the drought tolerance and resistance to bacterial infection is increased as compared to a plant that lacks said overexpression.

2. The method of claim 1, wherein the NHR1 gene is NHR1A or NHR1B.

3. The method of claim 1, wherein the plant is a monocotyledonous plant.

4. The method of claim 3, wherein the monocotyledonous plant is selected from the group consisting of corn, rice, wheat, sorghum, barley, oat, switchgrass, and turfgrass.

5. The method of claim 1, wherein the plant is a dicotyledonous plant.

6. The method of claim 5, wherein the dicotyledonous plant is selected from the group consisting of is a cotton, soybean, rapeseed, sunflower, tobacco, sugarbeet, and alfalfa.

7. The method of claim 1, wherein the plant has altered morphology as compared to a plant that lacks said overexpression.

8. The method of claim 7, wherein the altered morphology is reduced stomatal aperture.

9. The method of claim 1, wherein overexpressing of the NHR1 or GCN4 gene, or both, comprises expression of an exogenous NHR1 or GCN4 gene, or both.

10. The method of claim 1, wherein overexpressing of the NHR1 or GCN4 gene, or both, comprises expression of an endogenous NHR1 or GCN4 gene, or both.

11. A plant comprising overexpression of a NHR1 or GCN4 gene, or both, wherein the drought tolerance and resistance to bacterial infection is increased as compared to a plant that lacks said overexpression.

12. A seed that produces the plant of claim 11.

13. A seed produced by the plant of claim 11.

14. A DNA-containing plant part of the plant of claim 11.

15. The plant part of claim 14, further defined as a protoplast, cell, meristem, root, leaf, node, pistil, anther, flower, seed, embryo, stalk or petiole.

16. A method of producing a plant comprising increased drought tolerance and resistance to bacterial infection, the method comprising:
(a) obtaining a plant comprising overexpression of a NHR1 or GCN4 gene, or both, wherein the drought tolerance and resistance to bacterial infection is increased as compared to a plant that lacks said overexpression;
(b) growing said plant;
(c) crossing said plant with itself or another distinct plant to produce progeny plants; and
(d) selecting a progeny plant comprising overexpression of a NHR1 or GCN4 gene, or both, wherein said progeny plant comprises increased drought tolerance and resistance to bacterial infection as compared to a plant that lacks said overexpression.

17. A transgenic plant comprising a recombinant DNA molecule, wherein the recombinant DNA molecule overexpresses a NHR1 or GCN4 gene, or both, wherein said overexpression increases drought tolerance and resistance to bacterial infection.

18. The transgenic plant of claim 17, wherein the recombinant DNA molecule comprises a heterologous promoter operably linked to an exogenous NHR1 or GCN4 gene, or both.

19. The transgenic plant of claim 17, wherein the NHR1 gene is NHR1A or NHR1B.

20. The transgenic plant of claim 17, further defined as a legume.

21. The transgenic plant of claim 17, further defined as an R0 transgenic plant.

22. The transgenic plant of claim 17, further defined as a progeny plant of any generation of an R0 transgenic plant, wherein the transgenic plant has inherited the recombinant DNA molecule from the R0 transgenic plant.


SEQUENCE LISTING (11 sequences)

SEQ ID NO: 1 - DNA, 2085 bp, Arabidopsis thaliana
atgaccatcgtctctacttgttgtccttttggtctctctaatgtcactaagtgtatcaatttcgtgcagattaaagaagcaaaaacaatggtgaaatataatttcaagaagataacagttgtgccaaacggaaaacagtttgttgatatcgttctttcccgcactcagcggcagacaccaactgttgtccacaagggtgacaggatttgcaaactccgtagcttctacatgcggaaagtgaagttcacagaatcaaactttaacgagaagctctctgccattatcgacgagtttcctcgcctcaaggaaattcaacccttctatgaagatcttcttcatgtgctttacaataaagatcactacaagctcgctcttggtcaagtcaatactgcgaagaacaagatcagcaaaatcgccatggattatgtgaagctattgaagcatggtgattctctgtaccgatgcaagtgcttaaaagtggctgctcttgggcgtatgtgtactgttatgaaaggtattggtcctagtttggcttatcttgaacaggttaggcaacacattgcaaggcttccttcgattgatccaaacactcgaactctcttgatctgtggatgtcctaatgtgggaaagagttcttttatgaacaaagttacaagagctgacgtagctgttcagccatatgctttcacaacaaagtcactctttcttggtcatactgattacaagtgtttgaggtatcaggtgattgatacaccggggcttttggatagggaaattgaagaccgtaatatcattgaattgtgtagcatcaccgcgttggctcatatccgagctgctgttttgttctttcttgatatttcgggttcttgtggttacaccattgctcagcaggcttctctttttcacaacataaagtctgtgttcaagaacaaaccgttggtgattgtctgcaacaagactgatttgatgcctatggagaacttatctgaagaagataggaaacttattgaagagatgaaagatgaagctatgaagactgaaatgggagcaagtgaagaagcagtgattttggagatgagtactttgactgaagaaggtgtgatgtcagtgaggaatgctgcttgacagaggctgttagatcagagggtagcggcgaagatgaaatcgaaaaagatcggtgatcacttgaacaggttccatgtggcgatgccgaagagtcgtgatgaccaagaaaggctcccttgtatacctcaggtagctggtgaaaaggagaagcgaaagaccgagaaggatttggaagatgaaaatggtggagctggggtttattctgctagtctaaagaagaactacattttggctaaagaagaatggaaggatgatataatacctgagatctgtgactgtcataatgttgctgattttgttgattcagacatcttgaataggcttgaggaattgacaagtgaagaaagtcttaggaaggcagaggaagaagaagttggttttgagattgaaggcgaagaacttacagagaaggagaagaatgatttggctgctattcgtaagaagaaagctttgcttattgaagagagtagacttaagaagagcaatgcacaaaacagagcagctgttccaagaaagtttgacaaggataagaagtttacaaggaagagaatgggtagagagttatcatctcttggtcttgatccgtcttctgctttgaaccgtgcaagaagcaagtctagaggcagaaagagggaaaggtatgatgatttgagtaacgatgcaatggatgtggatgtgaatgatgatgagcaacataagaagaagaagatgtgtctgagatctaaatcaagatctttgtcaagatcaagatcagtgtcaaggccaccacatgaggttgtacctggtgaaggcttcaaagattcttcacagaagattaaggcaattaagattggacacaaatctcacagaaagagggacaaggctgcacgtcgtggagaagcagacagagttataccatctcttaaaccaaaacacttgttttcagggaaaagaggcaatggaaaaaaccaaaggcgttga

SEQ ID NO: 2 - DNA, 2016 bp, Arabidopsis thaliana
atggttcaatataatttcaagaggatcacagttgttcccaatgggaaggagttcgttgacatcatcctttcacggactcagcgtcagacaccaactgttgtccacaagggttacaagattaaccgtctccgtcagttctacatgagaaaggttaagtacactcagaccaacttccatgcgaagctctctgcaatcattgacgagtttcctcgccttgaacaaatccatcctttctatggtgatcttcttcatgtgctttacaataaagatcattacaagcttgctttaggccaagtgaacaccgccaggaacttgattagcaaaatctccaaggattatgtgaagctactcaagtatggtgattctttgtaccgctgcaagtgtcttaaggttgctgctcttggtcgtatgtgcactgttttgaagcgaatcactcctagtttggcttatctcgagcagatcaggcagcacatggctaggcttccttccatcgatcctaacactcgtactgtcttgatttgtggttaccctaatgttggcaagagttcttttatgaacaaggtcaccagagctgatgttgacgtccagccttatgctttcaccaccaaatccctctttgttggtcatactgattacaagtatctcaggtaccaagtcattgatacccccgggattttggataggccctttgaggaccgcaacatcattgaaatgtgcagtatcactgccttggcccatcttcgagctgctgttttgttctttctcgacatttctggatcatgtggttacactattgctcagcaggctgctcttttccacagcatcaagtccctctttatgaacaaacccttggtcattgtctgcaacaagactgatttgatgcccatggaaaacatctctgaagaagatcggaagctgattgaagagatgaagtctgaagctatgaagactgcaatgggagcaagtgaagaacaagtgcttttgaagatgagcactctgacagacgaaggtgtcatgtctgtgaagaatgccgcatgtgagaggctgttagatcaaagggtagaggccaagatgaaatccaaaaagatcaacgaccacctaaacaggttccatgtggctatcccgaagcctcgtgatagcatagaaaggctcccttgcatacctcaggttgttctggaggctaaagctaaggaagcagctgcaatggagaaacgaaagactgagaaagatttggaagaggagaatggtggagctggagtttattctgctagtctaaagaagaactacatcttgcaacatgatgaatggaaggaagacattatgcctgagattctcgatggtcacaatgtggctgacttcattgatccagacattttgcagaggcttgcagagttggaacgtgaagaagggataagggaggctggggttgaagaagctgatatggagatggatattgaaaagctttcggatgagcagctgaagcagctttctgagattagaaagaagaaagctatactcattaaaaaccacaggctcaagaagaccgttgcacagaaccgatcaactgttccaagaaagtttgataaggacaagaagtacacaacaaagagaatgggtagggagttatcagctatgggacttgatccgtcttctgcaatggaccgtgcaagaagcaagtctagagggaggaagagggatcgatcagaagatgcgggtaatgatgctatggacgttgatgatgagcaacagtcgaacaagaagcagcgtgtgagatcaaagtctagagctatgtcgatatcaagatctcagtcgaggcctcctgcacatgaagttgtgcctggtgaaggattcaaagactctactcagaagttatcggccattaagattagcaataaatctcacaaaaagagagacaagaatgcacgtcgtggtgaagctgacagagttataccaacgcttagaccgaaacatttgttttcaggcaagagagggaaaggaaaaaccgacaggcgttga

SEQ ID NO: 3 - DNA, 25 bp, Artificial sequence
  Primer
atggtgaaatataatttcaagaaga

SEQ ID NO: 4 - DNA, 19 bp, Artificial sequence
  Primer
acgcctttggttttttcca

SEQ ID NO: 5 - DNA, 21 bp, Artificial sequence
  Primer
atggttcaatataatttcaag

SEQ ID NO: 6 - DNA, 17 bp, Artificial sequence
  Primer
acgcctgtcggtttttc

SEQ ID NO: 7 - protein, 687 aa, Arabidopsis thaliana
MTIVSTCCPFGLSNVTKCINFVQIKEAKTMVKYNFKKITVVPNGKQFVDIVLSRTQRQTPTVVHKGDRICKLRSFYMRKVKFTESNFNEKLSAIIDEFPRLKEIQPFYEDLLHVLYNKDHYKLALGQVNTAKNKISKIAMDYVKLLKHGDSLYRCKCLKVAALGRMCTVMKGIGPSLAYLEQVRQHIARLPSIDPNTRTLLICGCPNVGKSSFMNKVTRADVAVQPYAFTTKSLFLGHTDYKCLRYQVIDTPGLLDREIEDRNIIELCSITALAHIRAAVLFFLDISGSCGYTIAQQASLFHNIKSVFKNKPLVIVCNKTDLMPMENLSEEDRKLIEEMKDEAMKTEMGASEEAVILEMSTLTEEGVMSRLLDQRVAAKMKSKKIGDHLNRFHVAMPKSRDDQERLPCIPQVAGEKEKRKTEKDLEDENGGAGVYSASLKKNYILAKEEWKDDIIPEICDCHNVADFVDSDILNRLEELTSEESLRKAEEEEVGFEIEGEELTEKEKNDLAAIRKKKALLIEESRLKKSNAQNRAAVPRKFDKDKKFTRKRMGRELSSLGLDPSSALNRARSKSRGRKRERYDDLSNDAMDVDVNDDEQHKKKKMCLRSKSRSLSRSRSVSRPPHEVVPGEGFKDSSQKIKAIKIGHKSHRKRDKAARRGEADRVIPSLKPKHLFSGKRGNGKNQRR

SEQ ID NO: 8 - protein, 671 aa, Arabidopsis thaliana
MVQYNFKRITVVPNGKEFVDIILSRTQRQTPTVVHKGYKINRLRQFYMRKVKYTQTNFHAKLSAIIDEFPRLEQIHPFYGDLLHVLYNKDHYKLALGQVNTARNLISKISKDYVKLLKYGDSLYRCKCLKVAALGRMCTVLKRITPSLAYLEQIRQHMARLPSIDPNTRTVLICGYPNVGKSSFMNKVTRADVDVQPYAFTTKSLFVGHTDYKYLRYQVIDTPGILDRPFEDRNIIEMCSITALAHLRAAVLFFLDISGSCGYTIAQQAALFHSIKSLFMNKPLVIVCNKTDLMPMENISEEDRKLIEEMKSEAMKTAMGASEEQVLLKMSTLTDEGVMSVKNAACERLLDQRVEAKMKSKKINDHLNRFHVAIPKPRDSIERLPCIPQVVLEAKAKEAAAMEKRKTEKDLEEENGGAGVYSASLKKNYILQHDEWKEDIMPEILDGHNVADFIDPDILQRLAELEREEGIREAGVEEADMEMDIEKLSDEQLKQLSEIRKKKAILIKNHRLKKTVAQNRSTVPRKFDKDKKYTTKRMGRELSAMGLDPSSAMDRARSKSRGRKRDRSEDAGNDAMDVDDEQQSNKKQRVRSKSRAMSISRSQSRPPAHEVVPGEGFKDSTQKLSAIKISNKSHKKRDKNARRGEADRVIPTLRPKHLFSGKRGKGKTDRR

SEQ ID NO: 9 - DNA, 2172 bp, Arabidopsis thaliana
atgggtaagaagaagtcagacgagagtgctgctaccacaaaggtgaagccaagtgggaaagatgcttcgaaagattctaaaaaagagaaattgtcagtctcggctatgcttgcaggcatggatcagaaagatgataaaccgaagaagggctcatcatctagaaccaaggctgctccgaaatctacatcttacactgatggcatagatcttcctccttctgatgaagaagacgacggtgaatctgatgaggaagagagacagaaggaagcaaggaggaagctgaagagtgaacaaaggcaccttgagatatctgtgactgataaggaacaaaagaagcgagaggcgaaagaaagattagctcttcaggctgcagagtcggcaaagagggaggctatgaaggacgatcatgatgcattcacggttgttattggaagcaagacctcagtgcttgaaggagacgacatggctgatgcaaatgttaaggatattaccatagaatctttttctgtatctgctcgaggtaaagagcttttgaagaatgcttctgtcaggatttcacatggtaaaaggtatgggttgatcgggccaaacggaatgggaaagtctacactgttaaagcttttagcttggaggaagattccagtgccaaagaatattgatgttcttcttgttgagcaagaggtggttggtgatgaaaagagtgctctgaatgcagttgtctctgccaatgaagagttggttaagctacgggaagaggctgaagctctgcagaagtcgtcttctggagctgatggagaaaatgttgatggtgaggatgatgatgatactggagaaaagcttgctgaactgtatgacaggctgcagattttagggtcagatgctgctgaagcacaggcatccaaaattcttgcggggttaggtttcacaaaagatatgcaagtgcgtgcgactcagtccttcagtggtggctggaggatgcgaatatcattagctagagctctgttcgtgcaacctacccttttgctgttagatgaacccactaaccatcttgacctgagagctgttctatggttagaggagtatttgtgtcgctggaagaagacactagttgttgtttcacatgaccgggacttcctcaacacagtctgcacggagataatacatctccatgaccaaaatctccacttctaccgtggtaatttcgatggttttgaaagcggatatgagcagcgtcgcaaggagatgaacaaaaaatttgatgtctacgacaaacagatgaaagcagcgaagaggactggaaaccggggtcaacaggagaaggtaaaggacagggccaagtttactgctgcaaaagaagcatccaagagtaagtcaaagggcaagacagtggatgaagaaggcccagcaccagaagctccaaggaagtggagagattacagtgtggtgttccacttcccagaaccaactgagctcactcctcctcttctgcagttaattgaggttagcttcagctatcccaacaggccagatttcagactctcgaatgttgatgtaggtatcgatatggggacacgggttgcgatagttgggcctaacggagcaggaaagtccactctattaaatcttcttgcgggagatttagttccaacagagggtgaaatgagaagaagccagaagctgaggattggcaggtattctcagcattttgttgaccttttaacaatgggggaaacaccggttcagtatctccttcgtcttcatcctgaccaagagggatttagcaagcaagaggcagtgcgagcgaagctaggcaagtttgggctaccaagtcacaatcacttatctccaattgcgaaattgtctggaggacaaaaggctagggttgtgttcacctcgatctcaatgtcaaaaccacacattttgcttctggacgagcctacaaatcacttagacatgcagagtatagatgccttggcggatgcactagatgagttcacaggtggagttgtgttggtgagtcacgactcgagactcatatcacgtgtatgtgcggaagaggagaagagtcaaatttgggttgtagaagacggaacagtgaatttcttcccaggcacatttgaagagtacaaagaagatctccaaagagaaatcaaagcagaagttgatgagtga

SEQ ID NO: 10 - protein, 723 aa, Arabidopsis thaliana
MGKKKSDESAATTKVKPSGKDASKDSKKEKLSVSAMLAGMDQKDDKPKKGSSSRTKAAPKSTSYTDGIDLPPSDEEDDGESDEEERQKEARRKLKSEQRHLEISVTDKEQKKREAKERLALQAAESAKREAMKDDHDAFTVVIGSKTSVLEGDDMADANVKDITIESFSVSARGKELLKNASVRISHGKRYGLIGPNGMGKSTLLKLLAWRKIPVPKNIDVLLVEQEVVGDEKSALNAVVSANEELVKLREEAEALQKSSSGADGENVDGEDDDDTGEKLAELYDRLQILGSDAAEAQASKILAGLGFTKDMQVRATQSFSGGWRMRISLARALFVQPTLLLLDEPTNHLDLRAVLWLEEYLCRWKKTLVVVSHDRDFLNTVCTEIIHLHDQNLHFYRGNFDGFESGYEQRRKEMNKKFDVYDKQMKAAKRTGNRGQQEKVKDRAKFTAAKEASKSKSKGKTVDEEGPAPEAPRKWRDYSVVFHFPEPTELTPPLLQLIEVSFSYPNRPDFRLSNVDVGIDMGTRVAIVGPNGAGKSTLLNLLAGDLVPTEGEMRRSQKLRIGRYSQHFVDLLTMGETPVQYLLRLHPDQEGFSKQEAVRAKLGKFGLPSHNHLSPIAKLSGGQKARVVFTSISMSKPHILLLDEPTNHLDMQSIDALADALDEFTGGVVLVSHDSRLISRVCAEEEKSQIWVVEDGTVNFFPGTFEEYKEDLQREIKAEVDE

SEQ ID NO: 11 - DNA, 2133 bp, Oryza sativa
atgggccgcaaagatacttcctcgtcgtcctccgccgccggcggcaagaaggacaagccgatgtcggtctccgccattctggcctccatggacgcgccggcgtccaaggccaagccatccaaggcggcgtccaagcccaagccctccaaggcgcccgcgtcgtcttacatgggcgacatcgacctgcccccctccgatgaggaggaggacgacgccgacctcgttgccatggccacgaagcccaaggcggcccgcgccaccgtcgacctcaacgccatcgcgccctcgcagaaggacgcgaagaagaaggacaagcgcgaggcgatggcggccgcgcaagccgaggcggccaagcaggaggcgctccgcgacgaccgcgacgcattctctgtcgttattggcgcgcgcgtcgctggatccgccggggcctccgagggtgactccgccgccgccgatgacaacatcaaggatattgtgctcgagaacttctctgtttctgcccgcgggaaggagctactcaagaacgcctcgctccggatctcgcacggccggcgctatggcctcgtcgggcccaacggcatgggcaagtccacccttctgaagctgctgtcatggcggcaggtcccggtgccccggagcattgatgtcctgcttgtggagcaggaaattattggagacaatcgttcagcgctcgaggctgttgttgctgctgatgaagagctcgcagcgcttcgtgccgagcaggcaaagctcgaggcctctaacgatgctgatgacaatgagcggcttgctgaggtttatgagaagctcaacctccgggattctgatgctgcccgggctcgtgcatccaagatccttgctgggctggggtttgatcaggctatgcaggccaggtccactaaatcgttcagtggtggctggaggatgcgcatctcgcttgctcgtgcgctgttcatgcagccaacattgttgctgcttgatgaaccgactaatcatcttgacctccgagctgttctttggttggagcaatacttgtgctcacagtggaagaaaacactgattgttgtgtcccatgatcgcgacttcctgaacacagtttgcaatgagatcattcatttgcatgataagaatctgcatgtttatcgtggaaattttgacgactttgagagtgggtatgaacagaagaggaaggagatgaataggaagtttgaggtgtttgaaaagcagatgaaggcagcaaagaagactgggagcaaggcagcgcaagacaaggtcaaaggtcaggcactatcaaaggctaataaggaggctgccaagagcaaggggaagggaaagaatgtagcaaatgatgacgacgacatgaagccagctgatcttccacagaaatggcttgactacaaggttgagttccacttcccagagccaactttgctcacaccaccgctccttcagctcattgaggtgggcttcagctaccctaataggccagatttcaagctgtctggtgttgatgttggcattgacatgggaacacgtgttgcaattgttggtcccaatggggcaggaaaatctacacttcttaatttacttgctggtgatcttaccccaaccaaaggagaggtaaggaggagtcagaagctgaggattgggcgatactcacagcattttgttgacttactgacaatggaggaaaatgcagttcagtatttgctcaggctccatcctgatcaggagggaatgagcaaagcagaggctgtacgtgcgaagctcggaaaatttggtttaccagggcacaaccatctcactccaatagttaaattatctggtggacagaaggcccgtgttgtgttcacttcaatatcaatgtcacatccccacattctcctgctggatgagccaacaaatcacctagacatgcaaagtatcgatgcattggcagatgcactggatgaattcactggtggtgtggtcttggttagccatgactcgagattgatctctagagtgtgtgacgatgagcagaggagtgagatatgggttgtagaagatggcactgtgaacaaatttgatggaacatttgaggactacaaggatgaacttttggaagaaatcaaaaaggaagttgaagagtaa